SEQUENCE LISTING (23 sequences)

SEQ ID NO: 1 - protein, 272 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 2 - protein, 272 aa, synthetic construct
  REGION 1..272: note = IGFBP5_265/269 mutant
  VARIANT 265: note = Xaa is one of Gly, Ala, Val, Leu, Ile, Pro, Phe, Met, and Trp
  VARIANT 269: note = Xaa is one of Gly, Ala, Val, Leu, Ile, Pro, Phe, Met, and Trp
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHXFDSXNVE

SEQ ID NO: 3 - protein, 272 aa, synthetic construct
  REGION 1..272: note = IGFBP5 Thr265 mutant
  VARIANT 265: note = Xaa is one of Gly, Ala, Val, Leu, Ile, Pro, Phe, Met, and Trp
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHXFDSSNVE

SEQ ID NO: 4 - protein, 272 aa, synthetic construct
  REGION 1..272: note = IGFBP5 Ser269 mutant
  VARIANT 269: note = Xaa is one of Gly, Ala, Val, Leu, Ile, Pro, Phe, Met, and Trp
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSXNVE

SEQ ID NO: 5 - DNA, 819 bp, Homo sapiens
  source 1..819: mol_type = genomic DNA
atggtgttgctcaccgcggtcctcctgctgctggccgcctatgcggggccggcccagagcctgggctccttcgtgcactgcgagccctgcgacgagaaagccctctccatgtgcccccccagccccctgggctgcgagctggtcaaggagccgggctgcggctgctgcatgacctgcgccctggccgaggggcagtcgtgcggcgtctacaccgagcgctgcgcccaggggctgcgctgcctcccccggcaggacgaggagaagccgctgcacgccctgctgcacggccgcggggtttgcctcaacgaaaagagctaccgcgagcaagtcaagatcgagagagactcccgtgagcacgaggagcccaccacctctgagatggccgaggagacctactcccccaagatcttccggcccaaacacacccgcatctccgagctgaaggctgaagcagtgaagaaggaccgcagaaagaagctgacccagtccaagtttgtcgggggagccgagaacactgcccacccccggatcatctctgcacctgagatgagacaggagtctgagcagggcccctgccgcagacacatggaggcttccctgcaggagctcaaagccagcccacgcatggtgccccgtgctgtgtacctgcccaattgtgaccgcaaaggattctacaagagaaagcagtgcaaaccttcccgtggccgcaaacgtggcatctgctggtgcgtggacaagtacgggatgaagctgccaggcatggagtacgttgacggggactttcagtgccacaccttcgacagcagcaacgttgagtga

SEQ ID NO: 6 - DNA, 48 bp, synthetic construct
  misc_feature 1..48: note = gene for IGFBP5 shRNA #443
  source 1..48: mol_type = other DNA
aggctgaagcagtgaagaaggctcgagccttcttcactgcttcagcct

SEQ ID NO: 7 - DNA, 48 bp, synthetic construct
  misc_feature 1..48: note = gene for IGFBP5 shRNA #467
  source 1..48: mol_type = other DNA
gcagaaagaagctgacccagtctcgagactgggtcagcttctttctgc

SEQ ID NO: 8 - DNA, 48 bp, synthetic construct
  misc_feature 1..48: note = gene for IGFBP5 shRNA #674
  source 1..48: mol_type = other DNA
acaagagaaagcagtgcaaacctcgaggtttgcactgctttctcttgt

SEQ ID NO: 9 - DNA, 31 bp, synthetic construct
  misc_feature 1..31: note = forward primer for amplifying IGFBP5 wildtype plasmid
  source 1..31: mol_type = other DNA
ataagaatgcggccgcatggtgttgctcacc

SEQ ID NO: 10 - DNA, 29 bp, synthetic construct
  misc_feature 1..29: note = reverse primer for amplifying IGFBP5 wildtype plasmid
  source 1..29: mol_type = other DNA
cggaattctcactcaacgttgctgctgtc

SEQ ID NO: 11 - DNA, 21 bp, synthetic construct
  misc_feature 1..21: note = Sense region_targeting 443-463 region
  source 1..21: mol_type = other DNA
aggctgaagcagtgaagaagg

SEQ ID NO: 12 - DNA, 21 bp, synthetic construct
  misc_feature 1..21: note = antisense region_targeting 443-463 region
  source 1..21: mol_type = other DNA
ccttcttcactgcttcagcct

SEQ ID NO: 13 - DNA, 58 bp, synthetic construct
  misc_feature 1..58: note = forward primer for amplifying pLKO-puro.1 vetor which is encoding shRNA targeting 443-463 region of IGFBP5
  source 1..58: mol_type = other DNA
ccggaggctgaagcagtgaagaaggctcgagccttcttcactgcttcagccttttttg

SEQ ID NO: 14 - DNA, 58 bp, synthetic construct
  misc_feature 1..58: note = reverse primer for amplifying pLKO-puro.1 vetor which is encoding shRNA targeting 443-463 region of IGFBP5
  source 1..58: mol_type = other DNA
aattcaaaaaaggctgaagcagtgaagaaggctcgagccttcttcactgcttcagcct

SEQ ID NO: 15 - DNA, 21 bp, synthetic construct
  misc_feature 1..21: note = Sense region_targeting 467-487 region
  source 1..21: mol_type = other DNA
gcagaaagaagctgacccagt

SEQ ID NO: 16 - DNA, 21 bp, synthetic construct
  misc_feature 1..21: note = antisense region_targeting 467-487 region
  source 1..21: mol_type = other DNA
actgggtcagcttctttctgc

SEQ ID NO: 17 - DNA, 58 bp, synthetic construct
  misc_feature 1..58: note = forward primer for amplifying pLKO-puro.1 vetor which is encoding shRNA targeting 467-487 region of IGFBP5
  source 1..58: mol_type = other DNA
ccgggcagaaagaagctgacccagtctcgagactgggtcagcttctttctgctttttg

SEQ ID NO: 18 - DNA, 58 bp, synthetic construct
  misc_feature 1..58: note = reverse primer for amplifying pLKO-puro.1 vetor which is encoding shRNA targeting 467-487 region of IGFBP5
  source 1..58: mol_type = other DNA
aattcaaaaagcagaaagaagctgacccagtctcgagactgggtcagcttctttctgc

SEQ ID NO: 19 - DNA, 21 bp, synthetic construct
  misc_feature 1..21: note = Sense region_targeting 674-694 region
  source 1..21: mol_type = other DNA
acaagagaaagcagtgcaaac

SEQ ID NO: 20 - DNA, 21 bp, synthetic construct
  misc_feature 1..21: note = antisense region_targeting 674-694 region
  source 1..21: mol_type = other DNA
gtttgcactgctttctcttgt

SEQ ID NO: 21 - DNA, 58 bp, synthetic construct
  misc_feature 1..58: note = forward primer for amplifying pLKO-puro.1 vetor which is encoding shRNA targeting 674-694 region of IGFBP5
  source 1..58: mol_type = other DNA
ccggacaagagaaagcagtgcaaacctcgaggtttgcactgctttctcttgttttttg

SEQ ID NO: 22 - DNA, 58 bp, synthetic construct
  misc_feature 1..58: note = reverse primer for amplifying pLKO-puro.1 vetor which is encoding shRNA targeting 674-694 region of IGFBP5
  source 1..58: mol_type = other DNA
aattcaaaaaacaagagaaagcagtgcaaacctcgaggtttgcactgctttctcttgt

SEQ ID NO: 23 - skipped

(19) United States
(12) Patent Application Publication
(10) Pub. No.: US 2026/0209298 A1
(43) Pub. Date: Jul. 23, 2026

(54) METHOD FOR PREVENTING OR TREATING CANCER BY BLOCKING EXCESSIVE PRODUCTION OF PHOSPHORYLATED IGFBP5

(71) Applicant: INDUSTRY-UNIVERSITY COOPERATION FOUNDATION HANYANG UNIVERSITY ERICA CAMPUS, Ansan-si (KR)

(72) Inventors: Hyungshin YIM, Seoul (KR); Jung-Won PARK, Seoul (KR); Da-Eun KIM, Ansan-si (KR)

(21) Appl. No.: 19/360,677

(22) Filed: Oct. 16, 2025

Related U.S. Application Data
(63) Continuation of application No. PCT/KR2024/001453, filed on Jan. 31, 2024.

(30) Foreign Application Priority Data
Apr. 13, 2023 (KR) 10-2023-0048846
Apr. 13, 2023 (KR) 10-2023-0048932

(51) Int. Cl. C07K 14/47; A61K 38/00; A61P 35/04; C12N 15/113; C12N 15/86

(52) U.S. Cl. CPC C07K 14/4743; A61P 35/04; C12N 15/113; C12N 15/86; A61K 38/00; C12N 2740/15043

(57) ABSTRACT

The present invention is to confirm that PLK1 induces tumor formation and cancer metastasis through IGFBP5 phosphorylation, and provide an IGFBP5 mutant in which a region phosphorylated by PLK1 is mutated and a vector expressing same. The IGFBP5 mutant of the present invention maintains a binding ability to PLK1, and thus binds to PLK1 overexpressed competitively with wild-type IGFBP5 in cancer cells, thereby making it possible to inhibit tumor formation and cancer metastasis, and the provision of a vector expressing the mutant in cancer cells can inhibit cancer mobility and invasiveness and inhibit tumor formation. The mutant of the present invention is safe because it does not affect the inherent function of IGFBP5 in normal cells, and thus can be useful in the treatment of various diseases caused by abnormal cell growth, especially degenerative diseases such as primary and metastatic solid cancer and leukemia.

### CROSS-REFERENCE TO RELATED APPLICATION

This application is a bypass continuation of pending PCT International Application No. PCT/KR2024/001453, which was filed on Jan. 31, 2024, and which claims priority to and the benefit of Korean Patent Application No. 10-2023-0048846, which was filed in Korean Intellectual Property Office on Apr. 13, 2023, and Korean Patent Application No. 10-2023-0048932, which was filed in Korean Intellectual Property Office on Apr. 13, 2023, the disclosure of which are incorporated herein by reference in its entirety.

### SEQUENCE LISTING

The instant application contains a Sequence Listing which has been submitted electronically in XML format and is hereby incorporated by reference in its entirety. Said XML copy, created on Apr. 13, 2023, is named SQLISTING_HE25070MH_2026-03-19.xml and is 32485 bytes in size.

### TECHNICAL FIELD

The disclosure confirms that tumor protein kinase enzyme PLK1 induces tumor formation and cancer metastasis through IGFBP5 phosphorylation, and provides an IGFBP5 mutant in which a region phosphorylated by PLK1 is mutated and an shRNA effective for inhibiting IGFBP5 expression and the like. Specifically, the IGFBP5 mutant of the disclosure is a mutant protein in which an amino acid is substituted at a serine position 269 and a threonine position 265 of wild-type IGFBP5, and the IGFBP5 mutant and the IGFBP5 shRNA of the disclosure may reduce tumor formation, cancer motility, and invasiveness.

### BACKGROUND ART

IGFBP5 is known to regulate IGF-1 signaling by binding to IGF-I (Firth S M et al., Endocr Rev, (2002) 23(2002), pp. 824-854). IGF-1 is similar to insulin and is an important growth factor for tissue growth, development, and maintenance (Sahitya K Denduluri et al., Genes and Disease, 2(2015), pp. 13-25). IGFBP5, which is found in all vertebrates, is located on human chromosome 2, is expressed in various human tissues, and encodes a protein of approximately 29 KDa. IGFBP5 is composed of an N-terminal domain, an L-domain, and a C-terminal domain, and has a signal peptide that allows secretion to the extracellular matrix (ECM) and a nuclear localization sequence (NLS) that allows translocation to the nucleus (Mustafa Akkiprik et al., Breast Cancer Res, 212 (2008)). Both the N-terminal domain and the C-terminal domain of IGFBP5 have IGF-1 binding sites, and the C-terminal domain includes a binding site for ECM proteins (Hodgkinson S C et al., J Mol Endocrinol, 13 (1994), pp. 105-112). IGFBP5 is known to play a potential role in carcinogenesis through two pathways: an IGF-1 dependent pathway and an IGF-1 independent pathway (Mustafa Akkiprik et al., BMC cancer, 103 (2009)). IGFBP5 binds to IGF-1 to increase the half-life of IGF-1 and can serve as a reservoir for release when needed in tissues (Mohan S et al., J Endocrinol, 175 (2002), pp. 19-31). IGFBP-5 binds to both the cell surface and the extracellular matrix (ECM), and when IGFBP-5 binds to the ECM or heparin, the affinity of IGFBP-5 for IGF-I decreases by 8 to 17 fold and the biological action of IGF-I on cells is enhanced (Jones J I et al., J Cell Biol, 121 (1993), pp. 679-87). In the IGF-1 independent pathway, IGFBP5 can enter the cytoplasm through binding with a putative receptor. Cytoplasmic accumulation of IGFBP5 by interaction with other proteins exhibits an anti-apoptosis effect and stimulates metastasis, while IGFBP5 that enters the nucleus through binding with Importin-β increases apoptosis. IGFBP5 mRNA was detected in breast cancer tissue samples and showed high expression in tumor samples compared to adjacent normal tissues. Additionally, it was observed that the IGFBP5 protein was confined to the cytoplasm (Mustafa Akkiprik et al., Breast Cancer Res, 212 (2008)).

The expression of IGFBP5 is observed in malignant tumors including breast cancer (Mustafa Akkiprik et al., Breast Cancer Res, 212 (2008)), colorectal cancer (Yu Deng et al., International Journal of General Medicine, 15 (2022), pp. 6485-6497), pancreatic cancer (Sarah K et al., Molecular Carcinogenesis, 45 (2006), pp. 814-827), ovarian cancer (Graeme Walker et al., Clinical Cancer Research, 13 (2007), pp. 1438-1444), glioblastoma (Vani Santosh et al., Cancer Epidemiology Biomarkers & Prevention, 19 (2010), pp. 1399-1408), and non-functional pituitary adenoma (NEPA) (Francoise Galland, Endocrine-Related Cancer, 17 (2010), pp. 361-371).

In the clinical data of breast cancer, the expression of IGFBP5 was higher in T1 breast carcinoma compared to benign breast epithelium, and higher expression was observed in T1N1 compared to T1NO carcinoma. Although IGFBP5 may be one of the markers for predicting lymph node metastasis in T1 invasive breast cancer, and this suggests that the expression of IGFBP5 is associated with metastasis (Huamin Wang M D et al., The Breast journal, 14 (2008), pp. 261-267), the role of IGFBP5 in mediating EMT is not precisely known.

Polo-like kinase 1 (PLK1) is well known as a causative protein kinase enzyme of carcinogenesis (Barr et al., Nat Rev Mol Cell Biol 5 (2004) 429-440; Yim and Erikson, Mutation Research Reviews Mutation Research, 761 (2014) 31-39), and according to recent studies, the function of PLK1 as a kinase enzyme that induces metastasis has also been revealed (Cai et al., Am J Transl Res 8 (2016) 4172-4183; Wu et al., eLife 5 (2016) e10734; Shin et al., Oncogene, 39 (2020), pp. 767-785), and it is being studied as a target molecule for cancer treatment, and based on this, anticancer agent development research through the development of inhibitors against PLK1 is being competitively conducted centering on multinational companies (J. Van den Bossche, 36 (2016), pp. 749-786; Rosie Elizabeth Ann Gutteridge, Molecular Cancer Therapeutics, 15 (2016), pp. 1427-1435; Yim, Anti-Cancer Drugs 24(2013) 999-1006; Yim and Erikson, Mutation Research Reviews Mutation Research, 761 (2014) 31-39). PLK1 has a kinase active domain with kinase activity and a polo box domain that binds to substrates, and phosphorylation at the T210 position induces the activation of PLK1. The activated PLK1 phosphatase is an enzyme that phosphorylates Ser/Thr residues of substrates (Barr et al., Nat Rev Mol Cell Biol 5 (2004) 429-440). Functionally, the expression is increased in growing and dividing cells, particularly reaching a peak in expression and activity during the mitotic phase of the cell cycle. Therefore, it is known that the expression rate is also high in rapidly growing cancer cells (Yim, Anti-Cancer Drugs 24(2013) 999-1006; Yim and Erikson, Mutation Research Reviews Mutation Research, 761 (2014) 31-39), and it has been experimentally reported that in many cases, the expression increases during the stepwise malignant transformation process of cancer cells. In particular, it has been reported that the expression increases as the malignant stage becomes higher in non-small cell lung cancer, head and neck cancer, laryngeal cancer, breast cancer, liver cancer, endometrial cancer, colorectal cancer, ovarian cancer, pancreatic cancer, prostate cancer, and the like (Yim, Anti-Cancer Drugs 24(2013) 999-1006; Yim and Erikson, Mutation Research Reviews Mutation Research, 761 (2014) 31-39). According to recent research reports and the studies of the present inventors, it has been reported that active PLK1 is involved in increasing the metastatic potential of cancer (Cai et al., Am J Transl Res 8 (2016) 4172-4183; Wu et al., eLife 5 (2016) e10734; Shin et al., Oncogene, 39 (2020), pp. 767-785), and accordingly, the value of PLK1 as a molecular target for treating metastatic cancer has recently begun to be highlighted. Therefore, anticancer agents that target the functional regulation of phosphorylation substrates of PLK1 are expected to be effective not only for treating primary cancer but also for treating metastatic cancer.

Although metastatic cancer shows different characteristics depending on the cancer type, it can account for up to 90% of all cancer patient deaths, which demonstrates its risk level (Valastyan, S. and Weinberg, R. A., Cell 147 (2011) 275-292; Khan, I. and Steeg, P. S., Lab Invest 98 (2018) 198-210). In anticancer treatment, even if primary cancer is treated, if metastasis cannot be prevented, it is known that the survival rate becomes very low due to cancer recurrence and the like (Valastyan, S. and Weinberg, R. A., Cell 147 (2011) 275-292; Shibue, T. and Weinberg, Semin Cancer Biol 21 (2011) 99-106). In cancer treatment, inhibition of cancer cell proliferation and inhibition of metastasis are not effects that always appear together. Therefore, the development of treatments that can inhibit cancer metastasis can dramatically improve cancer treatment efficiency by increasing the survival rate of many cancer patients, and thus, the development of treatment targets or therapeutic agents that effectively inhibit cancer metastasis and invasion for the treatment of metastatic cancer is necessary.

### DISCLOSURE OF THE INVENTION

### Technical Goals

The disclosure relates to the use of an IGFBP5 protein containing point mutations as a therapeutic agent for metastatic cancer, wherein the IGFBP5 protein containing point mutations inhibits metastasis in solid cancers derived from various tissues, and utilizes a potent inhibitory action on metastatic potential, invasiveness, and selective tumor formation of cancer cells appearing in non-small cell lung cancer cells by the IGFBP5 protein containing IGFBP5 point mutations.

The disclosure also relates to the use as an apoptosis inducer, utilizing the action of sensitively inducing apoptosis in metastatic cancer cells, as well as general solid cancers. The disclosure may be usefully used for treating various solid cancers and metastatic cancers.

Accordingly, a technical problem to be achieved by the disclosure is to provide an IGFBP5 mutant in which a serine amino acid at position 269 is substituted and a vector expressing the same.

Additionally, a technical problem to be achieved by the disclosure is to provide an IGFBP5 mutant in which a threonine amino acid at position 265 is substituted and a vector expressing the same.

Furthermore, the disclosure aims to provide a pharmaceutical composition for cancer prevention or treatment, including the mutant or the vector as an active ingredient.

Additionally, the disclosure aims to confirm the reduction of metastatic potential, invasiveness and selective tumor formation of cancer cells through inhibition of IGFBP5 expression, and to provide shRNA effective for inhibiting IGFBP5 expression for cancer prevention or treatment use.

Furthermore, the disclosure aims to provide a pharmaceutical composition for cancer prevention or treatment, including the IGFBP5 shRNA and the IGFBP mutant protein as active ingredients.

However, technical goals to be achieved are not limited to those described above, and other goals not mentioned above are clearly understood by one of ordinary skill in the art from the following description.

### Technical Solutions

Wild-type IGFBP5 (insulin-like growth factor binding protein 5) is a protein consisting of an amino acid sequence of SEQ ID NO: 1, and is encoded by a base sequence of SEQ ID NO: 5.

To solve the above problem, the disclosure provides an IGFBP5 mutant protein in which an amino acid residue at position 265 and/or 269 of IGFBP5 of SEQ ID NO: 1, threonine, is substituted with glycine, alanine, valine, leucine, isoleucine, proline, phenylalanine, methionine, or tryptophan.

In an embodiment of the disclosure, the mutant protein may include or consist of an amino acid sequence of SEQ ID NO: 2.

In another embodiment of the disclosure, the mutant protein may include or consist of an amino acid sequence of SEQ ID NO: 3.

In another embodiment of the disclosure, an amino acid residue at position 265 of SEQ ID NO: 3 may be alanine.

In another embodiment of the disclosure, the IGFBP5 mutant protein may include or consist of an amino acid sequence of SEQ ID NO: 4.

In another embodiment of the disclosure, an amino acid at position 269 of SEQ ID NO: 4 may be alanine.

Furthermore, the disclosure provides a recombinant vector including a gene encoding the IGFBP5 mutant.

In another embodiment of the disclosure, the recombinant vector may be a viral vector, preferably a lentiviral vector.

Furthermore, the disclosure provides a pharmaceutical composition for cancer prevention or treatment, including the IGFBP5 mutant or the recombinant vector.

Furthermore, the disclosure provides a pharmaceutical composition for inhibiting cancer metastasis, including the IGFBP5 mutant or the recombinant vector.

The disclosure provides a pharmaceutical composition for cancer prevention or treatment, including an IGFBP5 expression inhibitor as an active ingredient.

In an embodiment of the disclosure, the IGFBP5 expression inhibitor may be an aptamer, siRNA, shRNA, or miRNA, including an oligonucleotide complementary to a part of IGFBP5 mRNA.

In another embodiment of the disclosure, the IGFBP5 expression inhibitor may be an oligonucleotide including a base sequence complementary to a base sequence of SEQ ID NO: 11, SEQ ID NO: 15, or SEQ ID NO: 19.

In another embodiment of the disclosure, the IGFBP5 expression inhibitor may be shRNA, including or consisting of any one base sequence of SEQ ID NO: 6 to SEQ ID NO: 8, but preferably may be shRNA, including or consisting of a base sequence of SEQ ID NO: 6.

Furthermore, the disclosure provides a pharmaceutical composition for cancer prevention or treatment, including the shRNA as an active ingredient.

In an embodiment of the disclosure, the composition may inhibit cancer metastasis by reducing motility and invasiveness of cancer cells.

In another embodiment of the disclosure, the cancer may be a solid cancer, and the solid cancer may be one or more cancers selected from the group consisting of glioblastoma, ovarian cancer, bladder cancer, non-functional pituitary adenoma, esophageal cancer, kidney cancer, breast cancer, stomach adenocarcinoma, rectal cancer, cervical cancer, endometrial cancer, lung cancer, and pancreatic cancer, wherein the lung cancer may be non-small cell lung cancer, and the non-small cell lung cancer may be particularly adenocarcinoma.

Furthermore, the disclosure provides a method of cancer prevention or treatment including administering the IGFBP5 mutant or the recombinant vector to a subject.

Furthermore, the disclosure provides a method of cancer prevention or treatment including administering to a subject the IGFBP5 expression inhibitor; and/or a recombinant vector including an IGFBP5 mutant or a gene encoding the same.

In an embodiment of the disclosure, the subject may be a human in need of cancer prevention or treatment, and may be a patient already having cancer and under treatment, or a patient for preventing metastasis after complete cure of cancer.

Furthermore, the disclosure provides a use of the IGFBP5 mutant, a recombinant vector including a gene encoding the same, or an IGFBP5 expression inhibitor for manufacturing a drug for cancer prevention or treatment.

### Effects of the Invention

The IGFBP5 mutant of the disclosure maintains a binding ability to PLK1, and thus binds to PLK1 overexpressed competitively with wild-type IGFBP5 in cancer cells, thereby making it possible to inhibit tumor formation and cancer metastasis, and the provision of a vector expressing the mutant in cancer cells can inhibit cancer mobility and invasiveness and inhibit tumor formation. The non-phosphorylated point mutant of the disclosure is safe because it does not affect the inherent function of IGFBP5 in normal cells, and thus can be useful in the treatment of various diseases caused by abnormal cell growth, especially degenerative diseases such as primary and metastatic solid cancer and leukemia.

### BRIEF DESCRIPTION OF DRAWINGS

FIGS. 1A to 1B are results of analyzing survival rates according to PLK1 and IGFBP5 expression in clinical analysis targeting adenocarcinoma patients.

FIG. 1A: Results of analyzing overall survival rates according to PLK1 and IGFBP5 expression in patients with lung adenocarcinoma at each stage using KM plotter.

FIG. 1B: Results of analyzing increased PLK1 and IGFBP5 expression in patients with lung adenocarcinoma at each stage through heat map analysis.

FIGS. 2A to 2G are results of observing IGFBP5 expression levels during mesenchymal transition by TGF-β.

FIG. 2A: Results of observing IGFBP5 expression using immunoblot when EMT occurred after treating A549 cells and H358 cells with 5 ng/ml TGF-β. To confirm whether EMT was well induced, the expression of epithelial marker E-cadherin and mesenchymal markers N-cadherin, Vimentin, and SNAI1 was also observed.

FIG. 2B: A graph numerically expressing immunoblot data observing IGFBP5 expression when EMT occurred after treating A549 cells and H358 cells with 5 ng/ml TGF-β.

FIG. 2C: Results of observing IGFBP5 mRNA level using real-time polymerase chain reaction (Real-time PCR) when EMT occurred after treating A549 cells and H358 cells with 5 ng/ml TGF-β.

FIG. 2D: Results of analyzing the expression of IGFBP5, PLK1, and p-PLK1 in lung fibroblast MRC5, primary lung cancer A549, and metastatic lung cancers H460, H358, and H1299 using immunoblotting.

FIG. 2E: Results of analyzing the expression of IGFBP5, PLK1, and p-PLK1 in lung fibroblast MRC5, primary lung cancer A549, and lung adenocarcinoma stage-specific cell lines (H522, H1373, H2009) using immunoblotting.

FIG. 2F: Results of observing IGFBP5 expression using immunoblot when EMT occurred after treating A549 cells and H358 cells with 5 ng/ml TGF-β. To confirm whether EMT was well induced, the expression of epithelial marker E-cadherin and mesenchymal markers N-cadherin, Vimentin, and SNAI1 was also observed.

FIG. 2G: A graph numerically expressing immunoblot data observing IGFBP5 expression when EMT occurred after treating A549 cells and H358 cells with 5 ng/ml TGF-β.

FIGS. 3A to 3C are results of analyzing survival rates of solid cancer patients according to IGFBP5 expression in clinical data, according to an embodiment of the disclosure.

FIG. 3A: Result graphs of analyzing survival rates according to IGFBP5 expression in cervical cancer (left graph), endometrial cancer (middle graph), and breast cancer (right graph) patients through KM PLOTTER analysis.

FIG. 3B: Result graphs of analyzing survival rates according to IGFBP5 expression in bladder cancer (left graph), esophageal cancer (middle graph), and ovarian cancer (right graph) patients through KM PLOTTER analysis.

FIG. 3C: Result graphs of analyzing survival rates according to IGFBP5 expression in kidney renal papillary cell carcinoma (left graph) and stomach adenocarcinoma (middle graph) patients through KM PLOTTER analysis.

FIGS. 4A to 4E is a result of analyzing the interaction between PLK1 and IGFBP5, phosphorylation of IGFBP5 by active PLK1, and phosphorylation sites.

FIG. 4A: Results of observing that IGFBP5 and PLK1 interact by performing immunoprecipitation using PLK1 antibody under conditions of treating A549 cells with 5 ng/ml TGF-β.

FIG. 4B: Results of observing that IGFBP5 and PLK1 interact by performing immunoprecipitation using PLK1 antibody under conditions of treating H460 cells with 2.5 ng/ml TGF-β.

FIG. 4C: Results of cloning IGFBP5 into pGEX-4T-1 vector tagged with GST to purify GST-labeled IGFBP5.

FIG. 4D: Results of purifying GST-tagged IGFBP5 using GST purification.

FIG. 4E: Results of performing phosphorylation enzyme assay using GST-labeled IGFBP5 together with active PLK1 (PLK1 TD).

FIGS. 5A to 5D are results of expressing non-phosphorylated point mutants to find sites of IGFBP5 phosphorylated by PLK1.

FIG. 5A: Results of identifying PLK1-phosphorylated IGFBP5 phosphorylation candidate sites through liquid chromatography mass spectrometry.

FIG. 5B: Results of substituting candidate sites of IGFBP5 with alanine in non-phosphorylated form using site-directed mutagenesis before performing phosphorylation enzyme assay.

FIG. 5C: Results of performing phosphorylation enzyme assay using non-phosphorylated point mutant proteins in which phosphorylation candidate sites of IGFBP5 were substituted with alanine using site-directed mutagenesis and active PLK1.

FIG. 5D: The results confirmed that threonine at position 265 and serine at position 269 are conserved in higher animals, including humans.

FIGS. 6A to 6C are results of observing the expression of EMT markers after expressing RFP-IGFBP5 through Tet-On 3G inducible system using lentivirus.

FIG. 6A: Results of cloning IGFBP5 into pLVX-TRE3G-eRFP vector to make lentivirus.

FIG. 6B: A diagram explaining Tet-On 3G inducible system, which is a system for RFP-IGFBP5 expression.

FIG. 6C: Results of observing IGFBP5 protein overexpression status and changes in epithelial markers (E-cadherin) and mesenchymal transition markers (Vimentin, N-cadherin) by immunoblot after expressing phosphorylated and non-phosphorylated point mutants of threonine at position 265 and phosphorylated and non-phosphorylated point mutants of serine at position 269 of IGFBP5 in lung cancer cells A549.

FIGS. 7A to 7D are experiments measuring the effect of overexpression of phosphorylated and non-phosphorylated point mutants of IGFBP5 on motility and invasiveness of cancer cells in lung cancer cells A549.

FIG. 7A: Results of observing RFP-IGFBP5 mRNA overexpression status and changes in epithelial marker (CDH1) and mesenchymal transition marker (CDH2, VIM, SNAI1) mRNA through real-time polymerase chain reaction (Real-time PCR) after expressing phosphorylated and non-phosphorylated point mutants of IGFBP5 in lung cancer cells A549.

FIG. 7B: Results of observing cell motility patterns through migration assay using inserts in lung cancer cells A549 expressing phosphorylated and non-phosphorylated point mutants of IGFBP5.

FIG. 7C: Results of observing cell invasiveness patterns through invasion assay using Matrigel and inserts in lung cancer cells A549 expressing phosphorylated and non-phosphorylated point mutants of IGFBP5.

FIG. 7D: A graph analyzing caspase-3 activity in total cancer cells expressing wild-type IGFBP5 or phosphorylated point mutants using Caspase-3 assay.

FIGS. 8A to 8B are experiments confirming metastatic potential of cancer cells expressing phosphorylated and non-phosphorylated point mutants of beta-catenin in a tail-vein-injection animal model.

FIG. 8A: Results of observing metastatic cancer formation effects to mouse lungs and metastasis inhibition effects by expression of non-phosphorylated point mutants of IGFBP5 when lung cancer cells expressing phosphorylated and non-phosphorylated point mutants of IGFBP5 were injected into mouse tail veins.

FIG. 8B: A graph numerically showing cancer formation frequency according to metastatic cancer occurrence effects and metastatic cancer occurrence inhibition effects in IGFBP5 non-phosphorylated point mutant expression lung cancer models when lung cancer cells expressing phosphorylated and non-phosphorylated point mutants of IGFBP5 were intravenously injected in mice.

FIGS. 9A to 9D are results of confirming metastasis inhibition effects of cancer cells and effects on cell survival by treatment with IGFBP5 shRNA, which is an IGFBP5 mRNA expression inhibitor, under metastatic conditions.

FIG. 9A: A graph measuring the degree of IGFBP5 expression inhibition by IGFBP5 shRNA according to target sequences using real-time polymerase chain reaction (Real-time PCR).

FIG. 9B: Results of observing protein-level epithelial-mesenchymal transition marker changes by IGFBP5 shRNA treatment in TGF-β-induced metastatic environment using immunoblot.

FIG. 9C: A graph numerically expressing immunoblot data observing protein-level epithelial-mesenchymal transition marker changes by IGFBP5 shRNA treatment in TGF-β-induced metastatic environment.

FIG. 9D: Results of observing mRNA-level epithelial-mesenchymal transition marker changes by IGFBP5 shRNA treatment in TGF-β-induced metastatic environment through real-time polymerase chain reaction (Real-time PCR).

FIGS. 10A to 10E are results of observing inhibition effects on motility and invasiveness of cancer cells and changes in survival rates by treatment with IGFBP5 shRNA, which is an IGFBP5 mRNA expression inhibitor, in metastatic environments induced by TGF-β of IGFBP5. It is also a result of observing motility and invasiveness of cancer cells by IGFBP5 phosphorylated point mutants.

FIG. 10A: Results of observing the effect of inhibiting cell motility by IGFBP5 shRNA treatment using Transwell in metastatic environments induced by TGF-β.

FIG. 10B: Results of observing the effect of inhibiting cell invasiveness by IGFBP5 shRNA treatment using Transwell and Matrigel in metastatic environments induced by TGF-β.

FIG. 10C: A graph analyzing caspase-3 activity changes by IGFBP5 shRNA treatment in TGF-β-induced metastatic environment using Caspase-3 assay.

FIG. 10D: Results of observing cell motility patterns through migration assay using inserts in lung cancer cells A549 expressing phosphorylated and non-phosphorylated point mutants of IGFBP5.

FIG. 10E: Results of observing cell invasiveness patterns through invasion assay using Matrigel and inserts in lung cancer cells A549 expressing phosphorylated and non-phosphorylated point mutants of IGFBP5.

FIGS. 11A to 11D are results of observing changes in survival rates of cancer cells by treatment with IGFBP5 shRNA, which is an IGFBP5 mRNA expression inhibitor, in solid cancers other than lung cancer.

FIG. 11A: A graph measuring the degree of IGFBP5 expression inhibition using real-time polymerase chain reaction (Real-time PCR) after treating cervical cancer cells HeLa and breast cancer cells BT-20 with IGFBP5 shRNA.

FIG. 11B: A graph analyzing caspase-3 activity changes by IGFBP5 shRNA treatment in HeLa cells and BT-20 cells using Caspase-3 assay.

FIG. 11C: A graph measuring IGFBP5 expression using real-time polymerase chain reaction (Real-time PCR) after treating cervical cancer cells HeLa with IGFBP5 shRNA in metastatic environments induced by TGF-β.

FIG. 11D: A graph analyzing caspase-3 activity changes by IGFBP5 shRNA treatment in TGF-β-induced metastatic environment using HeLa cells through Caspase-3 assay.

### BEST MODE FOR CARRYING OUT THE INVENTION

While researching PLK1 (Polo-like kinase 1), the present inventors confirmed that phosphorylation of IGFBP5 by PLK1 contributes to cancer metastasis, and as a result of exploring the phosphorylation region of IGFBP5 by PLK1 and inducing mutations therein, confirmed that cancer motility and invasiveness were remarkably reduced, and confirmed that when IGFBP5 expression is knocked down, cancer motility and invasiveness are remarkably reduced, thereby completing the disclosure.

From the above, the present inventors provide a pharmaceutical composition for cancer prevention or treatment, including an IGFBP5 inhibitor as an active ingredient.

In the disclosure, “IGFBP5 inhibitor” may lower overexpressed IGFBP5 in tumor tissues to normal expression levels, and more specifically means an agent that lowers IGFBP5 phosphorylated by PLK1 to the level of normal tissues. Specifically, an IGFBP5 inhibitor may reduce the expression level of IGFBP5 mRNA, and may be an IGFBP5 mutant that interacts with PLK1 but is not phosphorylated as a competitive inhibitor of wild-type IGFBP5.

PLK1 is known to be overexpressed in various cancer types, and research on PLK1 molecules as targets for cancer therapeutic development is being actively conducted. However, PLK1 is a kinase involved in various signaling pathways, such as the cell cycle, and the inhibition of PLK1 makes it difficult to exclude the possibility of other actions in addition to the effect of inhibiting tumor growth, and there is a problem that the original function of PLK1 cannot be performed smoothly in its action in normal cells.

Research on the interaction between PLK1 and IGFBP5 has not been reported at all.

To examine the clinical correlation between PLK1 and IGFBP5, the present inventors analyzed KM plotter data based on TCGA data to confirm the expression of PLK1 and IGFBP5 and patient survival rates in non-small cell lung cancer patients, and as a result, found clinical significance that the expression of PLK1 and IGFBP5 increases according to the cancer progression stage of non-small cell lung cancer patients and this lowers patient survival rates. Furthermore, heat map analysis of patients whose tumor tissues showed increased IGFBP5 expression compared to normal tissues confirmed that IGFBP5 expression was high in patient groups with advanced stages (Example 1).

Furthermore, to confirm quantitative changes in IGFBP5 under conditions where metastasis occurred, as a result of examining the expression of IGFBP5 protein and mRNA under conditions treated with TGF-β, it was observed that the expression of IGFBP5 increased together with EMT markers (Example 2). From the above, it can be seen that the expression of IGFBP5 increases according to cancer progression and epithelial-mesenchymal transition.

In addition, as a result of confirming whether solid cancers other than lung cancer also show the same IGFBP5 expression pattern as in lung cancer, the results confirmed that when IGFBP5 expression is high in patients with cervical cancer, endometrial cancer, breast cancer, bladder cancer, esophageal cancer, ovarian cancer, kidney cancer, and stomach adenocarcinoma, survival rates are low (Example 3).

Subsequently, the present inventors aimed to target downstream molecules in the mechanism by which PLK1 is involved in cancer proliferation and metastasis, and specifically, to confirm whether the interaction between PLK1 and IGFBP5 is induced under conditions that cause metastatic cancer, after inducing metastatic lung cancer with TGF-β, immunoprecipitation was performed to observe increased binding between PLK1 and IGFBP5 (Example 4).

In addition, IGFBP5 binds to IGF-I to regulate IGF-I signaling. The present inventors sought to devise a method that maintains the original function of IGFBP5 in normal cells but can block only its action at the cancer occurrence and metastasis stages. Since the present inventors observed clinically that PLK1 and IGFBP5 are overexpressed in non-small cell lung cancer, particularly as the metastatic stage is higher, if phosphorylation of IGFBP5 by PLK1 is assumed to be a necessary condition for cancer metastasis, cancer metastasis can be suppressed if only the phosphorylation of IGFBP5 by PLK1 is prevented, so the present inventors explored the phosphorylation region of IGFBP5 by PLK1 and selected Ser269 and Thr265 as phosphorylation candidate regions (Examples 4 and 5).

Subsequently, the present inventors produced lentiviruses expressing mutants in which serine at position 269 and threonine at position 265 were substituted with alanine (non-phosphorylated form) or aspartate (phosphorylated form), respectively, transformed cancer cells using the lentiviruses, and confirmed the expression of EMT markers in cancer cells expressing each mutant. As a result, it was confirmed that in cancer cells expressing mutants in which serine at position 269 and threonine at position 265 were substituted with alanine, the expression of epithelial markers increased and the expression of mesenchymal markers decreased, indicating that serine at position 269 and threonine at position 265 of IGFBP5 are effective as targets for blocking phosphorylation by PLK1 (Example 6).

More specifically, the present inventors conducted cancer cell migration assays to observe the metastatic potential of cancer cells in A549 cells expressing each protein of phosphorylated and non-phosphorylated point mutants of IGFBP5 through specific experiments. As a result of research, it was observed that in the experimental group expressing phosphorylated point mutants of IGFBP5, cancer cell motility increased even more than in the case of treatment with TGF-β (5 ng/ml), which is a positive control. Conversely, the motility of cancer cells expressing non-phosphorylated point mutant proteins was reduced (Example 7).

Furthermore, the present inventors conducted experiments on the invasiveness promotion and inhibition effects in cancer cells using phosphorylated point mutants and non-phosphorylated point mutants of IGFBP5 through specific experiments. Invasion assay using Matrigel was used to observe the invasiveness of cancer cells. First, to evaluate invasiveness, cells expressing each point mutant of IGFBP5 are dispensed together with serum-free medium into Matrigel inserts, and medium containing serum is dispensed into experimental plates and cultured for 5 days. Invaded cancer cells are stained with crystal violet for observation, dissolved in DMSO, and the absorbance is measured at a wavelength of 590 nm. As a result of research, it was observed that invasiveness increased compared to the control group in the lung cancer cell group expressing phosphorylated point mutant proteins of IGFBP5. In particular, high cancer cell invasiveness could be observed in phosphorylated point mutants of IGFBP5. On the other hand, it was observed that invasiveness decreased in the lung cancer cell group expressing non-phosphorylated point mutant proteins of IGFBP5. Therefore, the invasiveness promotion effect in cancer cells by phosphorylated point mutants of IGFBP5 and the invasiveness inhibition effect of cancer cells by non-phosphorylated point mutants could be observed in lung cancer cells (Example 7).

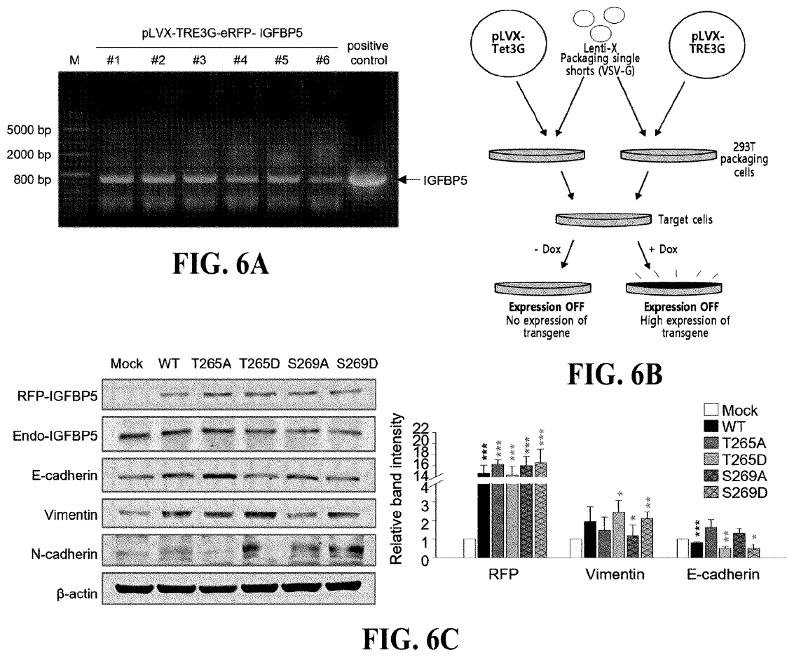

The disclosure conducted specific experiments to investigate cancer metastasis of proteins expressing PLK1-phosphorylated and non-phosphorylated point mutants of IGFBP5 in animal models by tail vein injecting A549 cells overexpressing each IGFBP5 mutant protein into BALB/c nude mice, raising them for 15 weeks, and performing laparotomy to observe cancer cell metastasis and tumor formation in organs (FIGS. 6A to 6C). In the lungs of animal groups injected with cells expressing phosphorylated point mutants of IGFBP5, cancer metastasis and tumor formation were observed more compared to control, wild-type, and cells expressing non-phosphorylated point mutants. On the other hand, in the lungs of animal groups injected with cells expressing non-phosphorylated point mutants of IGFBP5, it was confirmed that tumor formation ability was low. Therefore, the effect of promoting tumor formation of cancer cells by PLK1-phosphorylated point mutants of IGFBP5 at novel phosphorylation sites could be observed, and the tumor formation inhibition effect of cancer cells by non-phosphorylated mutant proteins of IGFBP5 was observed (Example 8).

Therefore, the disclosure can provide amino acids at positions 269 and 265 of IGFBP5 as targets for blocking phosphorylation of IGFBP5 by PLK1.

Subsequently, the present inventors sought to confirm whether cancer metastasis and invasion can be reduced through inhibition of IGFBP5 expression. For this purpose, shRNAs targeting nucleotide sequences at positions 443-463, 467-487, or 674-694 in the IGFBP5 mRNA sequence were designed and produced, and as a result of confirming each IGFBP5 expression inhibition efficacy, all shRNAs inhibited IGFBP5 expression, but particularly high IGFBP5 expression inhibition activity could be confirmed in shRNA targeting positions 443-463 of IGFBP5 mRNA (Example 9).

Using shRNA that effectively inhibits the expression of IGFBP5, it was confirmed whether inhibition of IGFBP5 expression can suppress the progression of epithelial-mesenchymal transition. As a result, it was confirmed that in lung cancer cells treated with the shRNA expression vector, epithelial-mesenchymal transition progression decreased even in a metastasis-inducing environment by TGF-β(Example 10). Furthermore, it was confirmed that the IGFBP5 shRNA remarkably reduced the motility and invasiveness of cancer cells even in a metastasis-inducing environment (Example 11).

Subsequently, the present inventors confirmed whether IGFBP5 can be used as a target for the treatment of other solid cancers in which IGFBP5 expression is increased, and as a result of treating cervical cancer cells with vectors expressing the shRNA, it was confirmed that the shRNA expression vectors promote apoptosis of cancer cells, and this was confirmed to appear identically even in a metastasis-inducing environment by TGF-β(Example 12).

Therefore, the disclosure provides a pharmaceutical composition for cancer prevention or treatment, including an IGFBP5 expression inhibitor as an active ingredient.

In the disclosure, “IGFBP5 expression inhibitor” is an oligonucleotide including a base sequence complementary to a part of IGFBP5 mRNA, and may be one or more selected from the group consisting of antisense oligonucleotides, aptamers, siRNA, shRNA, and miRNA that specifically bind to IGFBP5 mRNA.

In the disclosure, “antisense oligonucleotide” is DNA, RNA, or derivatives thereof containing a nucleic acid sequence complementary to the sequence of specific mRNA, and can act to inhibit translation of mRNA into protein by binding to complementary sequences within mRNA. An antisense oligonucleotide sequence means a DNA or RNA sequence that is complementary to the IGFBP5 sequence of SEQ ID NO: 1 and can bind to the sequence. For example, the antisense oligonucleotide may include or consist of a sequence complementary to any one sequence selected from the group consisting of sequences of SEQ ID NO: 3 and 4, but is not limited thereto. The length of antisense oligonucleotides may be 5 to 100 bases, 8 to 60 bases, 10 to 40 bases, or 10 to 30 bases. The antisense oligonucleotides can be synthesized in vitro by conventional methods and administered into living bodies, or can be administered in a form in which antisense oligonucleotides are synthesized in vivo. Methods for synthesizing antisense oligonucleotides in vitro may be carried out by biological/chemical methods according to conventional techniques in the technical field of the disclosure. Furthermore, the form that allows antisense oligonucleotides to be synthesized in vivo can be realized using recombinant vectors that express the antisense oligonucleotides, and such conventional methods can be easily implemented according to conventional methods in the technical field of the disclosure. Therefore, the design of antisense oligonucleotides of the disclosure can be easily produced according to methods known in the technical field of the disclosure, referring to the base sequence of SEQ ID NO: 1. For example, antisense oligonucleotides may include base sequences complementary to base sequences selected from the group consisting of sequences of SEQ ID NO: 3 or 4.

In the disclosure, “aptamer” is a single-stranded oligonucleotide, about 20 to 60 nucleotides in size, and means a nucleic acid molecule having binding activity for a predetermined target. Aptamers have various three-dimensional structures according to sequences and can have high affinity with specific substances, like antigen-antibody reactions. Aptamers can inhibit the activity of a predetermined target by binding to the predetermined target. The aptamers of the disclosure may be RNA, DNA, modified nucleic acids, or mixtures thereof, and may also be in linear or cyclic form. Preferably, the aptamers can play a role in inhibiting the expression or activity of IGFBP5 by binding to IGFBP5 mRNA. Such aptamers can be prepared by those skilled in the art from the sequence of IGFBP5 of SEQ ID NO: 1 by known methods.

In the disclosure, the terms “siRNA” and “shRNA” are nucleic acid molecules that can mediate RNA interference or gene silencing, and are used as efficient gene knockdown methods or gene therapy methods because they can suppress the expression of target genes. shRNA is formed into a hairpin structure by binding between complementary sequences within a single-stranded oligonucleotide, and in vivo, the shRNA is cleaved by dicer to become siRNA, which is a double-stranded oligonucleotide of small RNA fragments of 8 to 30 nucleotides in size, and can specifically bind to mRNA having complementary sequences to suppress expression. Therefore, which means of shRNA or siRNA to use can be determined by the choice of those skilled in the art, and if the mRNA sequences they target are identical, similar expression reduction effects can be expected. For the purpose of the disclosure, by specifically acting on IGFBP5 to cleave IGFBP5 mRNA molecules and induce RNA interference (RNAi) phenomena, the IGFBP5 can be suppressed. siRNA can be synthesized chemically or enzymatically. The method for producing siRNA is not particularly limited, and methods known in the art can be used. For example, there are methods for directly chemically synthesizing siRNA, methods for synthesizing siRNA using in vitro transcription, methods for cleaving long double-stranded RNA synthesized by in vitro transcription using enzymes, expression methods through intracellular delivery of shRNA expression plasmids or viral vectors, and expression methods through intracellular delivery of PCR (polymerase chain reaction)-induced siRNA expression cassettes, but are not limited thereto.

In the disclosure, “miRNA (micro RNA)” is a substance that naturally exists in cells and means a substance involved in the regulation of specific genes by inducing RNAi phenomena. Any miRNA that can suppress the expression or action of IGFBP5 of the disclosure is included in the disclosure without limitation to the type thereof.

Furthermore, the disclosure may provide IGFBP5 mutants in which S269 and/or T265 amino acids are substituted for cancer prevention and treatment uses.

In addition, PLK1 structurally includes an enzymatically active domain with kinase activity and a polo box domain that binds to substrates, and IGFBP5 mutants in which amino acids are substituted at S269 and T265 positions maintain binding affinity with PLK1. Therefore, mutants in which the phosphorylation region amino acids (amino acids at positions 269 and/or 265) are substituted will bind to PLK1 competitively with wild-type IGFBP5, and thus phosphorylation of wild-type IGFBP5 can be reduced in a mutant concentration-dependent manner.

The IGFBP5 mutants of the disclosure may be those in which S269 and T265 are substituted with amino acids that cannot be phosphorylated by PLK1, specifically glycine, alanine, valine, leucine, isoleucine, proline, phenylalanine, methionine, or tryptophan, and the present inventors confirmed the effect using mutants substituted with alanine.

The IGFBP5 mutants of the disclosure may be in a form included in carriers that enable efficient introduction into cells. The carriers are preferably vectors, and both viral vectors and non-viral vectors may be used. Viral delivery mechanisms include but are not limited to lentivirus, retrovirus, adenovirus, herpes virus, and avipox virus. Non-viral delivery mechanisms may include lipid-mediated transfection, liposomes, immunoliposomes, lipofectin, cationic surface amphiphiles, and combinations thereof. Lentivirus used as a viral vector for delivery of IGFBP5 shRNA and mutants in the disclosure is a type of retrovirus characterized by being able to infect not only dividing cells but also non-dividing cells due to the nucleophilicity of pre-integration complexes (viral “shells”) that enable active introduction into nucleopores or intact nuclear membranes.

Accordingly, the disclosure provides a recombinant vector including a gene encoding the IGFBP5 mutant, and the vector can be provided as a viral vector and used as a gene therapy-based anticancer agent.

In the disclosure, mutants mean those in which one or more amino acids constituting IGFBP5 protein are substituted, and unless otherwise stated, in the specification, mutants having anticancer effects by reducing cancer cell motility and invasiveness and promoting apoptosis of cancer cells mean those in which S269 and/or T265 amino acids of IGFBP5 are substituted.

The term ‘anticancer’ used in the specification means actions of suppressing or killing cancer cell proliferation and actions of suppressing or blocking cancer cell metastasis, meaning both cancer prevention and treatment, and can be used interchangeably. The term ‘prevention’ used in the specification means all actions of suppressing cancer formation or delaying onset by administration of compositions, and ‘treatment’ means all actions in which symptoms of the disease are improved or beneficially changed by administration of compositions.

The anticancer agents of the disclosure may additionally include other substances known to suppress phosphorylation of IGFBP5 or reduce its expression, such as compounds, natural products, novel proteins, and the like, in addition to the above-mentioned IGFBP5 expression inhibitors and IGFBP5 mutants.

The anticancer agents including IGFBP5 expression inhibitors, IGFBP5 mutants, and/or vectors expressing them of the disclosure may be used particularly for prevention and treatment of various solid cancers such as colon cancer, cervical cancer, endometrial cancer, bladder cancer, esophageal cancer, kidney cancer, stomach adenocarcinoma, ovarian cancer, breast cancer, and pancreatic cancer where IGFBP5 expression is excessive, may also be used for prevention and treatment of metastatic solid cancers induced by metastasis from primary cancers, and may be used for prevention of metastatic cancers.

The pharmaceutical compositions of the disclosure may include commonly used excipients, disintegrants, sweeteners, lubricants, or flavoring agents, and may be formulated into tablets, capsules, powders, granules, suspensions, emulsions, syrups, and other liquid forms by conventional methods. Specifically, the apoptosis activators of the disclosure may be formulated for oral administration as, for example, troches, lozenges, aqueous or oily suspensions, prepared powders or granules, emulsions, hard or soft capsules, syrups, or elixirs. At this time, for formulation into tablets and capsules, binders such as lactose, saccharose, sorbitol, mannitol, starch, amylopectin, cellulose, or gelatin; excipients such as dicalcium phosphate; disintegrants such as corn starch or sweet potato starch; and lubricants such as magnesium stearate, calcium stearate, sodium stearyl fumarate, or polyethylene glycol wax may be added. Furthermore, when formulated as capsules, in addition to the substances mentioned above, one or more pharmaceutically acceptable carriers can be included in the preparation. For example, pharmaceutically acceptable carriers include saline, sterile water, Ringer's solution, buffered saline, dextrose solution, maltodextrin solution, glycerol, ethanol, liposomes, and mixtures of one or more of these components, and if necessary, other conventional additives such as antioxidants, buffers, and bacteriostatic agents may be added. Furthermore, diluents, dispersants, surfactants, binders, and lubricants may be additionally added to formulate into injectable formulations such as aqueous solutions, suspensions, and emulsions, and antibodies or other ligands specific to target cells may be used in combination with the above carriers to enable specific action on target cells. Additionally, formulation can be preferably performed according to each disease or component using appropriate methods in the art or methods described in Remington's Pharmaceutical Science (Mack Publishing Company, Easton PA).

The compositions of the disclosure may be administered parenterally, and when administered parenterally, it is preferable to administer by intravenous injection, intramuscular injection, or intrathoracic injection infusion methods. For formulation for parenteral administration, the apoptosis-promoting anticancer agent can be mixed with water together with stabilizers or buffers to prepare solutions or suspensions, and these can be formulated into unit dosage forms of ampoules or vials.

The dosage is preferably selected according to the absorption, inactivation rate, and excretion rate of active ingredients in the body, the patient's age, sex, and condition, and the severity of the disease to be treated, and the apoptosis-promoting anticancer agent of the disclosure may be administered in divided doses of 1 to several times daily at a dosage of 1 to 100 mg, preferably 1 to 10 mg per kg of adult body weight.

In the disclosure, amino acid sequences are described using mixed abbreviations according to IUPAC-IUB nomenclature as follows:

Arginine (Arg, R), Lysine (Lys, K), Histidine (His, H), Serine (Ser, S), Threonine (Thr, T), Glutamine (Gln, Q), Asparagine (Asn, N), Methionine (Met, M), Leucine (Leu, L), Isoleucine (Ile, I), Valine (Val, V), Phenylalanine (Phe, F), Tryptophan (Trp, W), Tyrosine (Tyr, Y), Alanine (Ala, A), Glycine (Gly, G), Proline (Pro, P), Cysteine (Cys, C), Aspartic acid (Asp, D), Glutamic acid (Glu, E), Norleucine (Nle).

The disclosure can be subjected to various modifications and can have various embodiments, and thus, specific embodiments are illustrated in the drawings and described in detail in the following detailed description. However, this is not intended to limit the disclosure to specific embodiments, and it should be understood to include all modifications, equivalents, or substitutes that fall within the idea and scope of the disclosure. In the description of embodiments, detailed description of well-known related technologies will be omitted when it is deemed that such description will cause ambiguous interpretation of the disclosure.

### MODE FOR CARRYING OUT THE DISCLOSURE

Hereinafter, the disclosure will be described in detail by examples.

However, the following examples are merely illustrative of the disclosure, and the content of the disclosure is not limited by the following examples.

### EXAMPLES

### <Example 1> Confirmation of Correlation Between PLK1 and IGFBP5 Expression Levels and Survival Rates in Adenocarcinoma Through Clinical Data

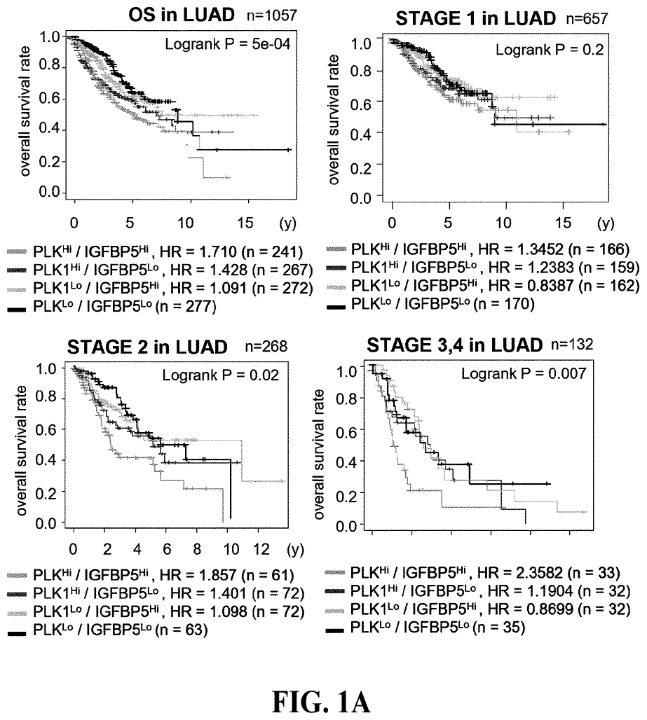

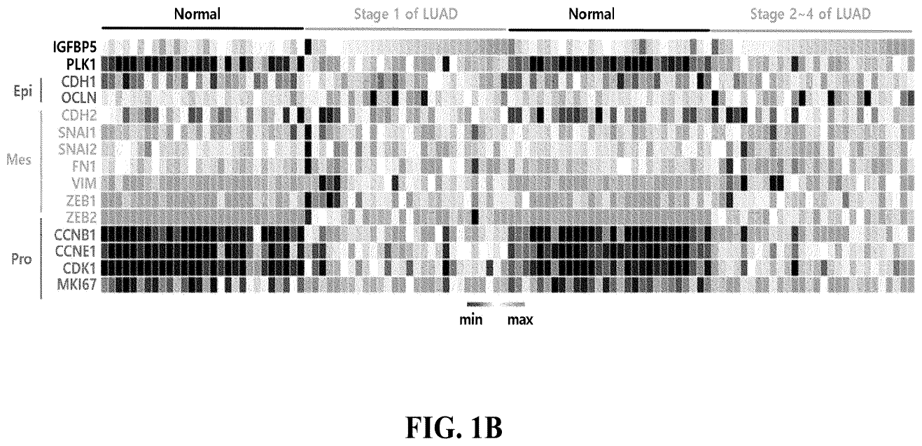

Survival rates according to PLK1 and IGFBP5 expression were analyzed in clinical analysis targeting adenocarcinoma patients (FIG. 1A). The higher the stage of adenocarcinoma patients, the greater the difference in survival rates between patients with high and low PLK1 and IGFBP5 expression. The number of patients with increased IGFBP5 expression in tumor tissues compared to normal tissues was analyzed through heat map analysis (FIG. 1B). It was confirmed that the expression of IGFBP5 and PLK1 was high in patient groups with high stages. From the above results, it can be seen that both factors are correlated with metastasis.

### <Example 2> Confirmation of IGFBP5 Expression in NSCLC Cells and Confirmation of IGFBP5 Expression Levels Under EMT Conditions Induced by TGF-β Treatment

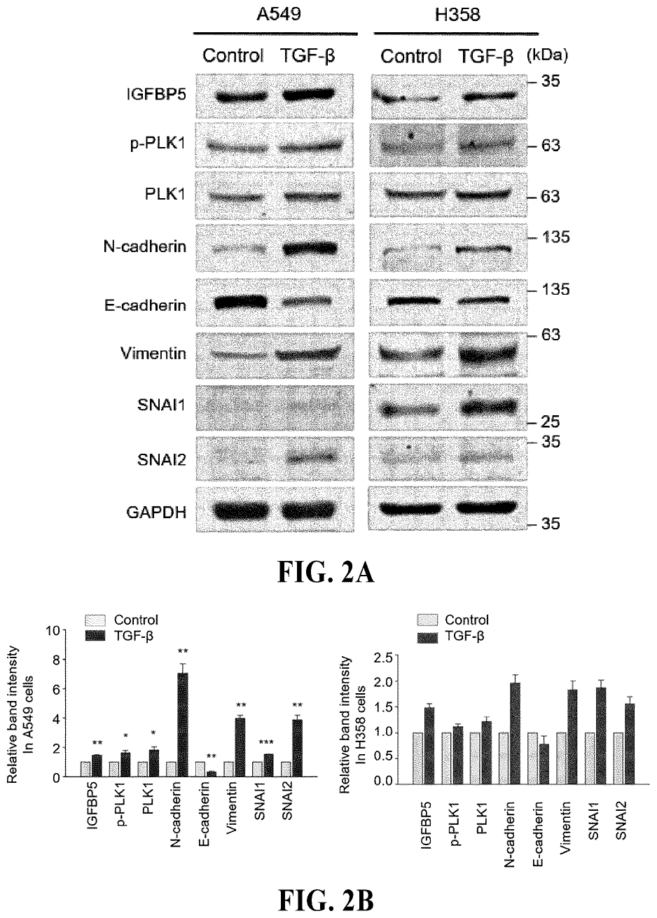

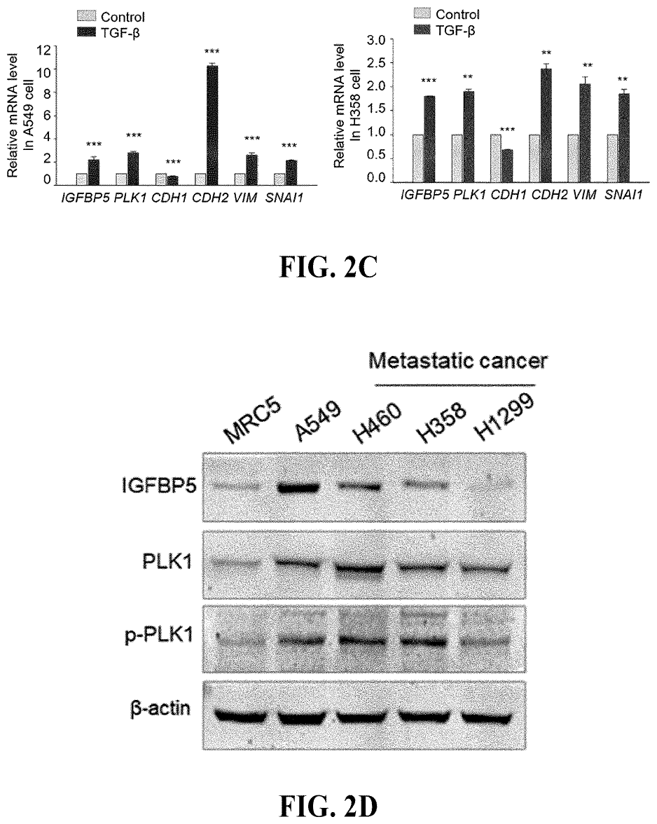

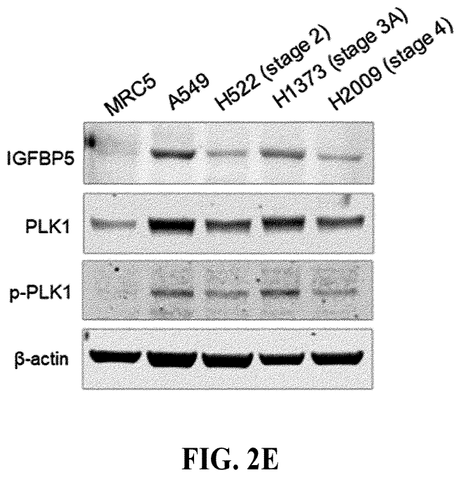

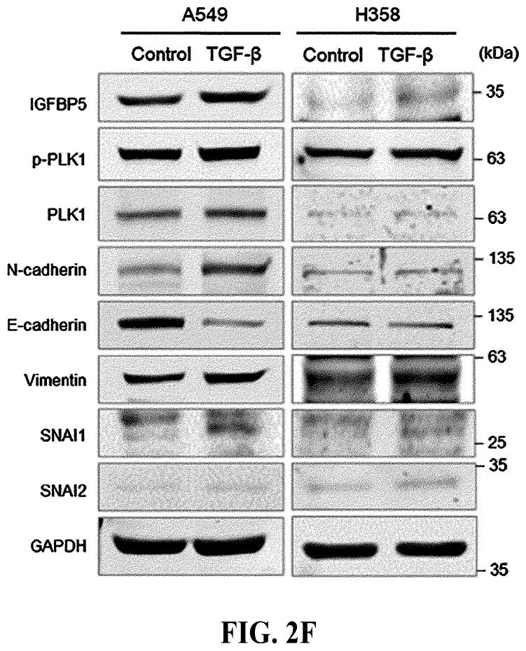

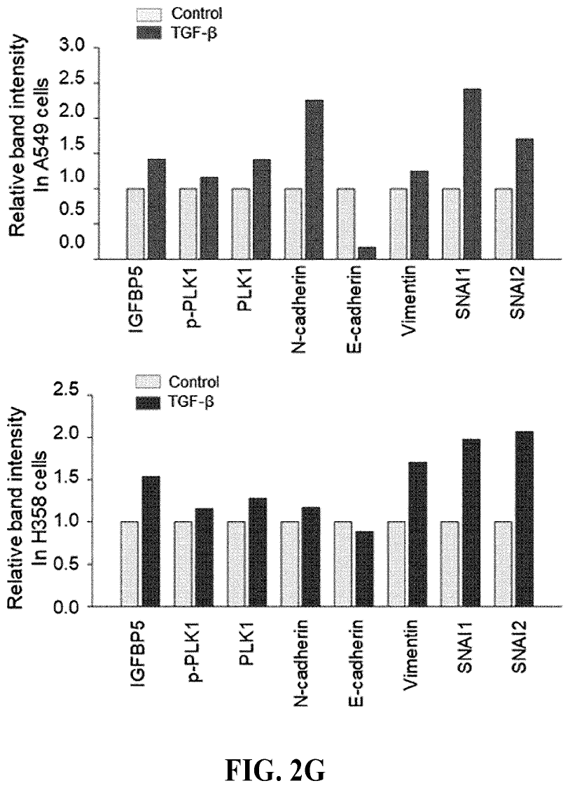

It was observed that IGFBP5 expression increased through immunoblot in lung cancer cell lines A549 and H358 treated with TGF-β to induce cancer metastasis (FIGS. 2A-2B). It was confirmed that IGFBP5 expression increased when TGF-β was treated. Real-time PCR was performed to confirm mRNA levels of IGFBP5 and EMT markers in lung cancer cell lines A549 and H358 treated with TGF-β to induce cancer metastasis (FIG. 2C). mRNA changes showed patterns similar to protein changes. From the above results, it can be seen that the expression level of IGFBP5 increases during epithelial-mesenchymal transition induced by TGF-β treatment.

Subsequently, the expression of IGFBP5, PLK1, and p-PLK1 was observed through immunoblot in non-small cell lung cancer cells. As a result of observing expression in normal fibroblast MRC5, primary lung adenocarcinoma cell A549, and metastatic lung cancer cells H460, H358, and H1299, it was confirmed that the expression of IGFBP5, PLK1, and p-PLK1 was higher in non-small cell lung cancer cells compared to MRC5 (FIG. 2D). The expression of IGFBP5, PLK1, and p-PLK1 was observed in normal fibroblasts, primary lung adenocarcinoma cell A549, stage 2 lung adenocarcinoma cell H522, stage 3A lung adenocarcinoma cell H1373, and stage 4 lung adenocarcinoma cell H2009. As a result of observation through immunoblot, IGFBP5 expression was highest in A549 and H1373, and it was confirmed that when IGFBP5 expression was high, the expression of PLK1 and p-PLK1 was also high (FIG. 2E). EMT was induced by treating A549 and H358 with TGF-β, and then IGFBP5 expression was observed. To confirm whether EMT was well induced, the expression of EMT markers was also confirmed together. After confirming that EMT was well induced, IGFBP5 expression was confirmed, and it was confirmed that expression increased when EMT occurred (FIG. 2F). Immunoblot data were expressed numerically and represented as graphs (FIG. 2G).

### <Example 3> Confirmation of Correlation Between IGFBP5 Expression Levels and Survival Rates in Solid Cancer Patients Other than Lung Cancer

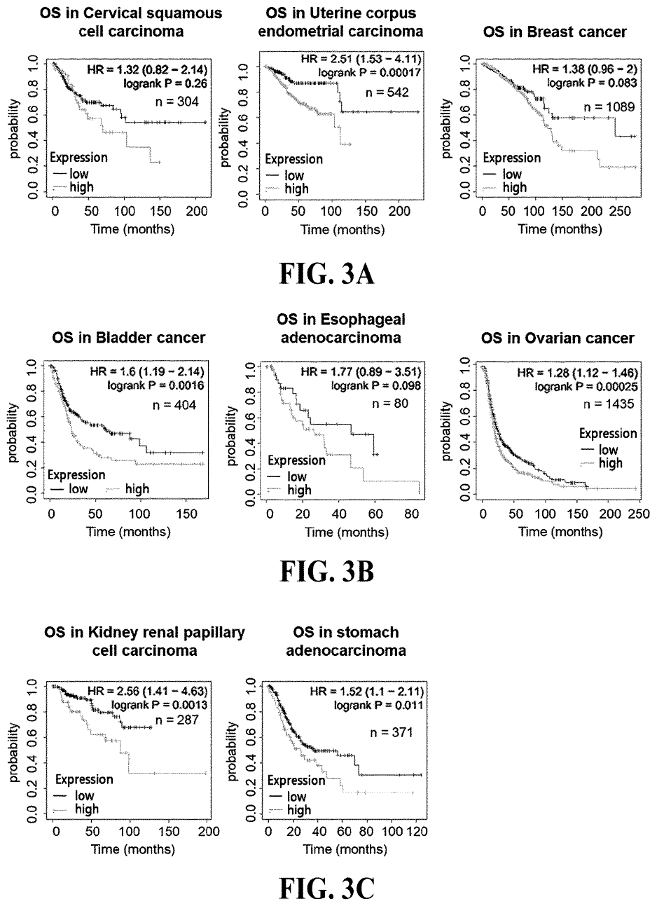

To confirm the clinical relevance of IGFBP5 in solid cancer patients other than lung cancer, the overall survival rate of patients according to IGFBP5 expression levels was confirmed through big data analysis (FIGS. 3A-3C). It was confirmed that the survival rate of patients with high IGFBP5 expression in cervical cancer, endometrial cancer, and breast cancer was significantly lower compared to the survival rate of patients with low IGFBP5 expression (FIG. 3A). Furthermore, when analyzing the survival rates of bladder cancer, esophageal cancer, and ovarian cancer patients, it was confirmed that patient survival rates were low when IGFBP5 expression was high (FIG. 3B). When analyzing the survival rates of kidney cancer and stomach adenocarcinoma patients, it was confirmed that they showed similar trends to other solid cancers (FIG. 3C).

### <Example 4> Confirmation of Phosphorylation of IGFBP5 by Active PLK1 in Metastatic Lung Cancer Cells Induced by TGF-β

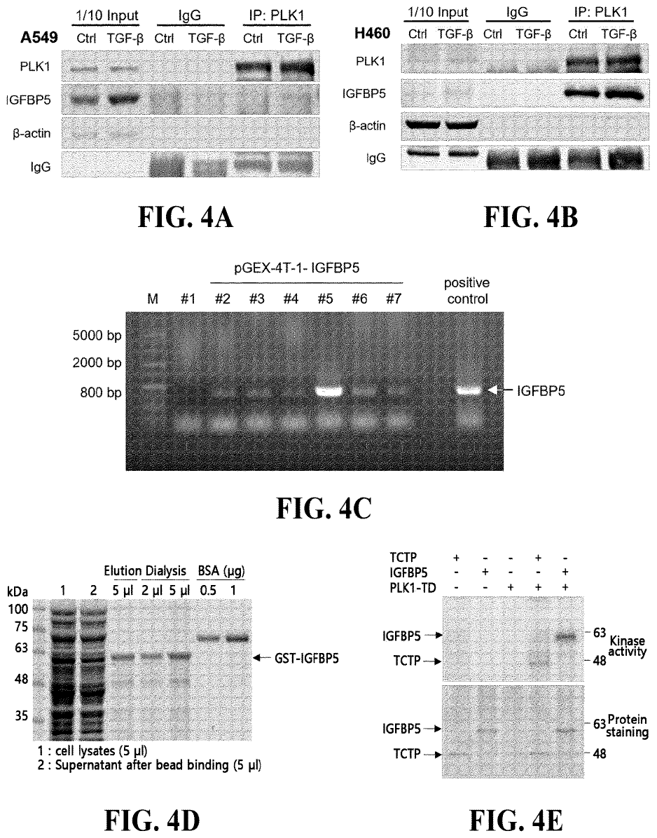

Immunoprecipitation was performed to explore the interaction between PLK1 and IGFBP5 under epithelial-mesenchymal transition conditions induced by TGF-β treatment. PLK1 protein was precipitated with agarose beads and PLK1 antibody and analyzed through immunoblot. In A549 cells, IGFBP5 bound to PLK1 only in the experimental group where cancer metastasis was induced by TGF-β. In H460 cells, IGFBP5 bound to PLK1 even in the control group, and in the experimental group where cancer metastasis was induced by TGF-β, the binding between PLK1 and IGFBP5 was increased (FIGS. 4A-4B). The above results suggest that the interaction between PLK1 and IGFBP5 is more active in cancer cells during the metastatic process.

Next, a kinase assay was performed to analyze the interaction method of the two proteins. To obtain GST-tagged IGFBP5 for performing a kinase assay, IGFBP5 was cloned into pGEX-4T-1 vector (FIG. 4C). GST-IGFBP5 was purified through GST-purification (FIG. 4D). As a method to prove whether PLK1, a serine/threonine kinase, phosphorylates IGFBP5 as a substrate, purified wild-type IGFBP5 and active PLK1-T210D were reacted together with radiolabeled γ-³²P-ATP and analyzed. IGFBP5 was phosphorylated by active PLK1-T210D more strongly than TCTP, which is a positive control (FIG. 4E).

### <Example 5> Confirmation of IGFBP5 Residues Phosphorylated by PLK1

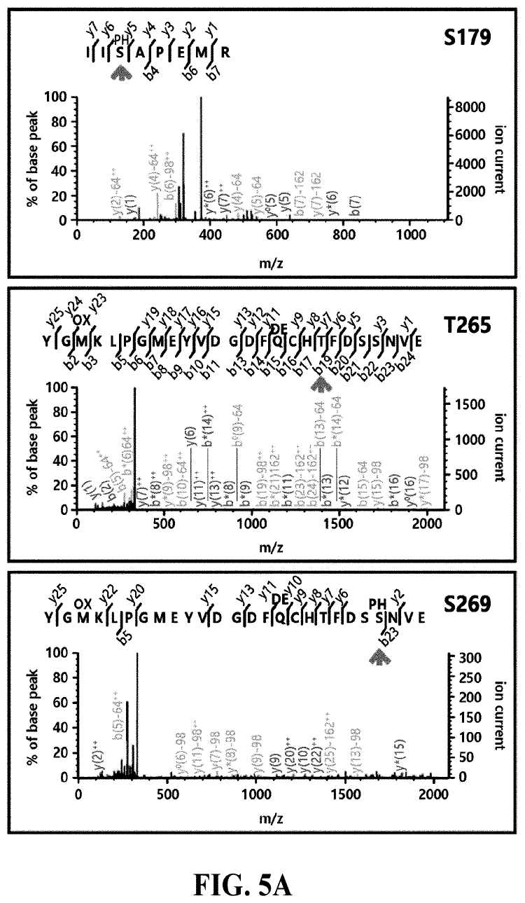

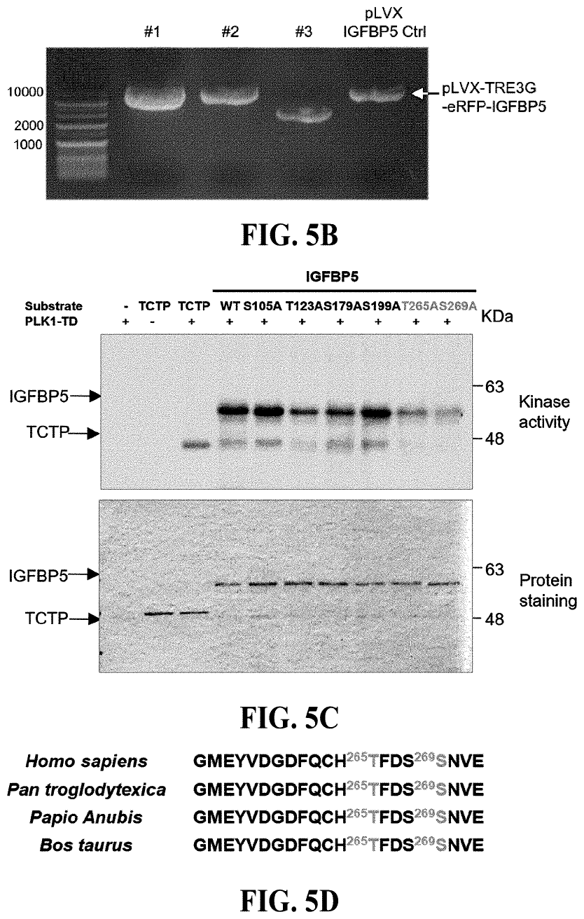

Liquid chromatography mass spectrometry was performed to confirm IGFBP5 residues phosphorylated by PLK1. Through this analytical method, Ser-179, Thr-265, and Ser-269 of IGFBP5 were predicted to be parts phosphorylated by PLK1 (FIG. 5A). Three predicted phosphorylation sites of IGFBP5 and IGFBP5 protein sequences were analyzed, and three sites similar to the PLK1 phosphorylation motif were produced as non-phosphorylated mutants substituted with alanine using site-directed mutagenesis (FIG. 5B).

The produced mutants were produced as GST-labeled proteins, purified, and a kinase assay was performed. Among the three predicted phosphorylation sites of IGFBP5, alanine mutations of Thr-265 and Ser-269 showed remarkably decreased phosphorylation levels compared to the wild type (FIG. 5C). From the above results, it was found that PLK1 interacts through phosphorylation reactions of IGFBP5 Thr265 and Ser269. As a result of confirming Thr265 and Ser269 of IGFBP5 in various species, it was confirmed that Thr265 and Ser269 of IGFBP5 are conserved in higher animals, including humans (FIG. 5D).

### <Example 6> Construction of Lentiviral System for Expression of Phosphorylated or Non-Phosphorylated Point Mutants of IGFBP5. Evaluation of Epithelial-Mesenchymal Transition Effects after Selection of Cells Expressing Phosphorylated and Non-Phosphorylated Point Mutant Genes of IGFBP5 Using Lentivirus

To construct a lentivirus system for expression of phosphorylated and non-phosphorylated genes of IGFBP5, the present inventors cut pLVX-TRE3G-eRFP with Not1 and EcoR1, amplified wild-type (WT) IGFBP5 plasmid by PCR using primers 5′-ATAAGAATGCGGCCGCATGGTGTTGCTCACC-3′ (forward primer, SEQ ID NO: 9) and 5′-CGGAATTCTCACTCAACGTTGCTGCTGTC-3′ (reverse primer, SEQ ID NO: 10), then cut the amplified product with Not1 and EcoR1, followed by subcloning into vector pLVX-TRE3G-eRFP (FIG. 6A). To construct a lentiviral system, pCMV-VSV.G, pCMV-Δ8.2, pLVX-TRE3G-eRFP-Target, or pLVX-Tet3G DNA were transfected to express them in HEK293 cells to express lentivirus, collect viruses, and use them for cancer cells. After transfection, virus culture medium was collected at 12-hour intervals until 72 hours, filtered through a 0.2 μm filter, and centrifuged at 17000 rpm, 4° C. for 90 minutes. The supernatant was discarded, viruses were collected with TNE Buffer, stored at 4° C., and used for infection of cancer cells from the next day.

RFP-IGFBP5 protein was expressed in A549 through Tet-On 3G inducible system using lentivirus (FIG. 6B). To examine the epithelial-mesenchymal transition regulatory effects of IGFBP5 proteins containing point mutations, cancer cells were cultured as follows to infect lung cancer cells with lentiviruses expressing phosphorylated and non-phosphorylated point mutants of IGFBP5.

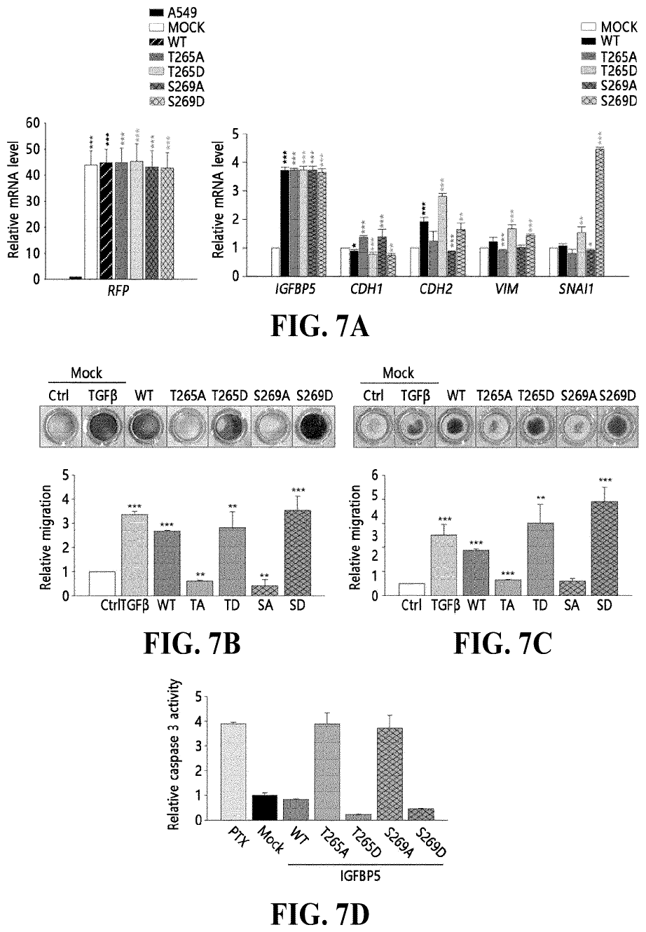

To construct stable cell lines expressing wild-type and phosphorylated and non-phosphorylated point mutants of IGFBP5 in lung cancer cell A549, pLVX-Tet3G-expressing lentivirus was first infected, and G418 was treated for 5 days to select infected cells. The selected A549Tet3G cells were infected with lentiviruses expressing wild-type and phosphorylated point mutants (T265D, S269D) and non-phosphorylated point mutants (T265A, S269A) of IGFBP5, then treated with puromycin for 48 hours to construct stable cell lines. Specifically, 5×10⁴ lung cancer cells A549 were dispensed, and the next day, 2 μg pCMV-VSV.G, 4 μg pCMV-Δ8.2, and 4 μg pLVX-TRE3G-eRFP-IGFBP5 were transfected to construct IGFBP5 overexpressing cells. The constructed cells were treated with 2 μg/ml doxycycline to induce expression of wild-type and phosphorylated and non-phosphorylated point mutants of IGFBP5, and then mRNA expression levels and protein expression levels were confirmed to verify whether each point mutant of IGFBP5 was well expressed. For comparison, EMT marker changes were observed in control group (Mock; lentivirus treatment group where target genes are not expressed), A549 lung cancer cell line administration group expressing IGFBP5 protein (WT), A549 lung cancer cell line administration group expressing phosphorylated point mutant IGFBP5 protein (T265D, S269D), and A549 lung cancer cell line administration group expressing non-phosphorylated point mutant IGFBP5 protein (T265A, S269A). As shown in FIG. 6C and FIG. 7A, it was confirmed through mRNA expression levels and protein expression levels that wild-type and each point mutant of IGFBP5 were well expressed. EMT marker expression was confirmed through immunoblot in lung cancer cells expressing phosphorylated point mutants of IGFBP5 and cells expressing non-phosphorylated point mutants of IGFBP5. In lung cancer cells expressing Thr265 phosphorylated point mutants of IGFBP5, E-cadherin expression decreased and Vimentin and N-cadherin expression increased compared to cells expressing non-phosphorylated point mutants of IGFBP5 (FIG. 6C). In lung cancer cells expressing Ser269 phosphorylated point mutants of IGFBP5, Vimentin and N-cadherin expression increased and E-cadherin expression decreased compared to cells expressing non-phosphorylated point mutants of IGFBP5 (FIG. 6C).

From the above results, it was suggested that phosphorylation of IGFBP5 in lung cancer cells has the effect of promoting epithelial-mesenchymal transition and is suppressed by non-phosphorylated point mutants.

### <Example 7> Confirmation of Metastatic Potential and Invasiveness Comparison of Cancer Cells Expressing Phosphorylated or Non-Phosphorylated Point Mutants of IGFBP5

EMT marker expression was confirmed through real-time PCR in lung cancer cells expressing phosphorylated point mutants of IGFBP5 and lung cancer cells expressing non-phosphorylated point mutants of IGFBP5. By confirming the expression of RFP and IGFBP5, it was confirmed that RFP-IGFBP5 expression was similar in all mutants (FIG. 7A). It was confirmed that CDH2, VIM, and SNAI1 expression increased and CDH1 expression decreased in cells expressing IGFBP5 phosphorylated point mutants compared to cells expressing IGFBP5 non-phosphorylated point mutants.

Cancer cell migration assays were performed to observe the effects of these point mutants on cancer cell motility in lung cancer cells A549 expressing wild-type, phosphorylated, or non-phosphorylated point mutant proteins of IGFBP5.

Specifically, 5×10⁴ lung cancer cells A549 expressing each wild-type, phosphorylated, or non-phosphorylated point mutant protein of IGFBP5 were dispensed into 8.0 μm, 24 well inserts, the medium containing 10% serum (FBS) was dispensed into 24 well plates, and inserts were added. For the positive control group, 0.5 ml of RPMI 1640 (10% FBS) containing 5 ng/ml TGF-β was dispensed and used. 48 hours after cell dispensing, 500 μl of 4% paraformaldehyde was dispensed, followed by washing 3 times with 1×PBS and staining with 0.05% crystal-violet solution for 5 minutes. After 5 minutes, the stained cells were washed 5 times with 1×PBS, and the extent of staining extent of staining was measured using Odyssey infrared imaging system. When the extent of staining extent of staining of the control group was set as 1, the relative extent of staining extent of staining in each experimental group was calculated and displayed as graphs.

As a result of research, in the experimental group expressing IGFBP5 phosphorylated point mutant proteins, the extent of staining extent of staining increased up to 5.5 times compared to the control group, similar to or higher than the TGF-β treatment group which is a positive control, and relatively, the non-phosphorylated point mutant experimental group showed lower extent of staining extent of staining compared to IGFBP5 phosphorylated point mutants (FIG. 7B). From the above results, it was found that IGFBP5 phosphorylated point mutants promote lung cancer cell motility, while IGFBP5 non-phosphorylated point mutants show the effect of suppressing lung cancer cell motility.

Invasion assays using Matrigel were performed to observe the effects of these point mutants on cancer cell invasiveness in lung cancer cells A549 expressing wild-type, phosphorylated, or non-phosphorylated point mutant proteins of IGFBP5.

Specifically, after completely melting Matrigel at 4° C. for 16-20 hours, Matrigel was diluted with cold serum-free RPMI 1640 (4° C.) to 1 mg/ml. 100 μl of Matrigel mixture (1 mg/ml) was added to 8.0 μm 24 well inserts and solidified in a 37° C. incubator for 12-20 hours. Lung cancer cells A549 expressing each wild-type, phosphorylated, or non-phosphorylated point mutant protein of IGFBP5 were diluted in serum-free RPMI 1640 (36° C.) at a cell number of 2×10⁵ cells/well and dispensed into each insert on solidified Matrigel inserts. 0.5 ml/well of warm RPMI 1640 (10% FBS) at 36° C. was dispensed into each well. For the positive control group, 0.5 ml of 36° C. RPMI 1640 (10% FBS) containing 5 ng/ml TGF-β was dispensed and used. Thereafter, the medium was changed every 3 days, and the degree of invasion was observed, and on the 5th day when cancer cell invasion was observed to have occurred sufficiently, the medium was removed and washed with 1×PBS, then cells inside the insert were scraped off with cotton swabs and washed with 1×PBS to remove any remaining cells and Matrigel inside the insert. 500 μl of 4% paraformaldehyde was dispensed into 24 wells with the outer surface of inserts, incubated at room temperature for 5 minutes, washed 3 times with 1×PBS, and stained with 0.05% crystal-violet solution for 5 minutes. After 5 minutes, the stained cells were washed 5 times with 1×PBS, the stain was solubilized in DMSO, and the absorbance at 590 nm was measured to assess the extent of staining. extent of staining extent of staining When the absorbance of the control group was set as 1, the relative absorbance in each experimental group was calculated and displayed as a graph.

As a result of research, the experimental group expressing IGFBP5 phosphorylated point mutant proteins increased 5.7 to 8 times compared to the control group, and relatively, the IGFBP5 non-phosphorylated point mutant experimental group showed lower relative absorbance compared to IGFBP5 phosphorylated point mutants (FIG. 7C). Therefore, it was found that IGFBP5 non-phosphorylated point mutants show the effect of suppressing lung cancer cell motility and invasiveness.

Cell survival rates were observed through caspase-3 assay in cells expressing IGFBP5 phosphorylated or non-phosphorylated point mutants. Caspase-3 activity greatly increased and cell survival rates decreased in Thr265 and Ser269 non-phosphorylated point mutants (FIG. 7D). From the above results, it was confirmed that when Thr265 and Ser269 of IGFBP5 are phosphorylated, cell survival increases, while IGFBP5 non-phosphorylated point mutants induce apoptosis according to caspase-3 activity.

### <Example 8> Evaluation of Cancer Cell Metastatic Potential by Proteins Containing Phosphorylated Sites and Non-Phosphorylated Point Mutations of IGFBP5 in Animal Models

The present inventors sought to evaluate the tumor formation promotion and inhibition effects of cancer cells using cancer cells expressing phosphorylated and non-phosphorylated point genes of IGFBP5 to observe the tumor formation ability of cancer cells in animal models.

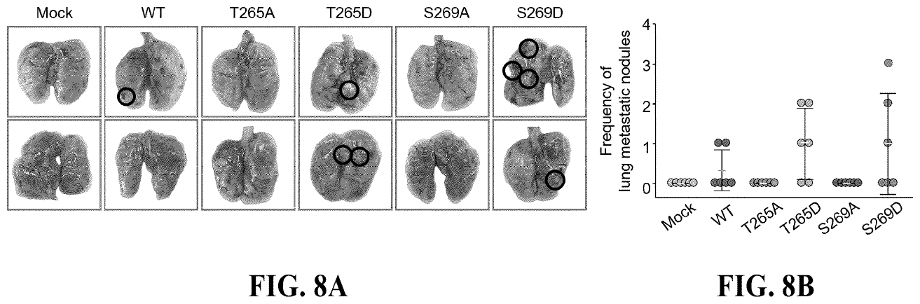

Specifically, A549 lung cancer cells (2×10⁶ cells) stably expressing IGFBP5 proteins containing point mutations were put in PBS and injected into mouse tail veins, raised for 15 weeks, and laparotomy was performed to observe cancer cell metastasis in organs. For comparison, cancer cell metastasis was examined in control group (Mock; lentivirus treatment group where target genes are not expressed), A549 lung cancer cell line administration group expressing IGFBP5 protein (WT), A549 lung cancer cell line administration group expressing phosphorylated point mutant IGFBP5 protein (T265D, S269D), and A549 lung cancer cell line administration group expressing non-phosphorylated point mutant IGFBP5 protein (T265A, S269A). Experiments were conducted on 6 mice for each experimental group, and the frequency of tumor formation, which is metastatic cancer in the lungs, was measured and displayed as a graph (FIGS. 8A-8B).

As shown in FIGS. 8A-8B, most tumors were formed in the lungs, so this experiment was also analyzed focusing on the lungs. In experimental groups administered with A549 cells expressing IGFBP5 proteins containing phosphorylated point mutations compared to the control group (Mock) and wild-type IGFBP5, relatively high tumor formation ability was observed. Particularly in T265D and S269D experimental groups among IGFBP5 phosphorylated point mutants, the highest tumor formation ability could be confirmed when observing the number of tumors formed. Conversely, in experimental groups administered with A549 cells expressing IGFBP5 proteins containing non-phosphorylated point mutations, relatively low tumor formation ability was observed (FIGS. 8A-8B).

### <Example 9> Selection of IGFBP5 shRNA

To confirm the mRNA expression inhibition effect of IGFBP5, shRNA and lentiviruses containing the shRNA were produced. Specifically, to suppress mRNA expression of IGFBP5, primers were produced to make shRNA targeting nucleotide sequences at positions 443-463, 467-487, or 674-694 in human IGFBP5 mRNA (Human IGFBP5 mRNA [NM_000599.4], SEQ ID NO: 5) sequence. For the 443-463 nucleotide target sequence sense region, 5′-AGG CTG AAG CAG TGA AGA AGG-3′ (SEQ ID NO: 11) was used, and for the antisense region, 5′-CCT TCT TCA CTG CTT CAG CCT-3′ (SEQ ID NO: 12) was used, on the basis of which the pLKO-puro.1-IGFBP5 plasmid was constructed using the pLKO-puro.1 vector. 5′-CCGG-AGGCTGAAGCAGTGAAGAAGG-CTCGAG-CCTTCTTCACTGCTTCAGCCT-TTTTTG-3′ (SEQ ID NO: 13) was used as forward primer, and 5′-AATTCAAAAA-AGGCTGAAGCAGTGAAGAAGG-CTCGAG-CCTTCTTCACTGCTTCAGCCT-3′ was used as reverse primer. For shRNA targeting 467-487 nucleotide sequence, 5′-GCA GAA AGA AGC TGA CCC AGT-3′ was used as the sense region, and 5′-ACT GGG TCA GCT TCT TTC TGC-3′ was used as the antisense region, on the basis of which the pLKO-puro.1-IGFBP5 plasmid was constructed using the pLKO-puro.1 vector. 5′-CCGG-GCAGAAAGAAGCTGACCCAGT-CTCGAG-ACTGGGTCAGCTTCTTTCTGC-TTTTTG-3′ (SEQ ID NO: 17) was used as the forward primer, and 5′-AATTCAAAAA-GCAGAAAGAAGCTGACCCAGT-CTCGAG-ACTGGGTCAGCTTCTTTCTGC-3′ (SEQ ID NO: 18) was used as the reverse primer. For shRNA targeting 674-694 nucleotide sequence, 5′-ACA AGA GAA AGC AGT GCA AAC-3′ (SEQ ID NO: 19) was used as the sense region, and 5′-GTT TGC ACT GCT TTC TCT TGT-3′ (SEQ ID NO: 20) was used as the antisense region, on the basis of which the pLKO-puro.1-IGFBP5 plasmid was constructed using the pLKO-puro.1 vector. 5′-CCGG-ACAAGAGAAAGCAGTGCAAAC-CTCGAG-GTTTGCACTGCTTTCTCTTGT-TTTTTG-3′ (SEQ ID NO: 21) was used as the forward primer, and 5′-AATTCAAAAA-ACAAGAGAAAGCAGTGCAAAC-CTCGAG-GTTTGCACTGCTTTCTCTTGT-3′ (SEQ ID NO: 22) was used as the reverse primer. The loop sequence is 5′-CTCGAG-3′ (SEQ ID NO: 23), and shRNA was produced in the form included in primers. The shRNA constructs were expressed through HEK293 cell transfection together with pHR′-CMV-VSVG and pHR′-CMV-deltaR8.2, and then cell culture medium was collected to produce lentiviruses. The lentiviruses were concentrated using a centrifuge. To confirm virus expression, A549 cells were cultured at 5×10⁴ cells/ml, and the next day lentiviruses were added at 20 ml/well to infection buffer (Infection Buffer; 10 mM HEPES (Sigma-Aldrich, USA), 1 mg/ml polybrene (Sigma-Aldrich, MO, USA)) to infect cancer cells. After 24 hours, puromycin (Sigma-Aldrich, MO, USA) was treated for 48 hours to select infected cells that survived, and RNA was taken from the selected cells to observe that IGFBP5 expression was suppressed through real-time polymerase chain reaction (Real-time PCR).

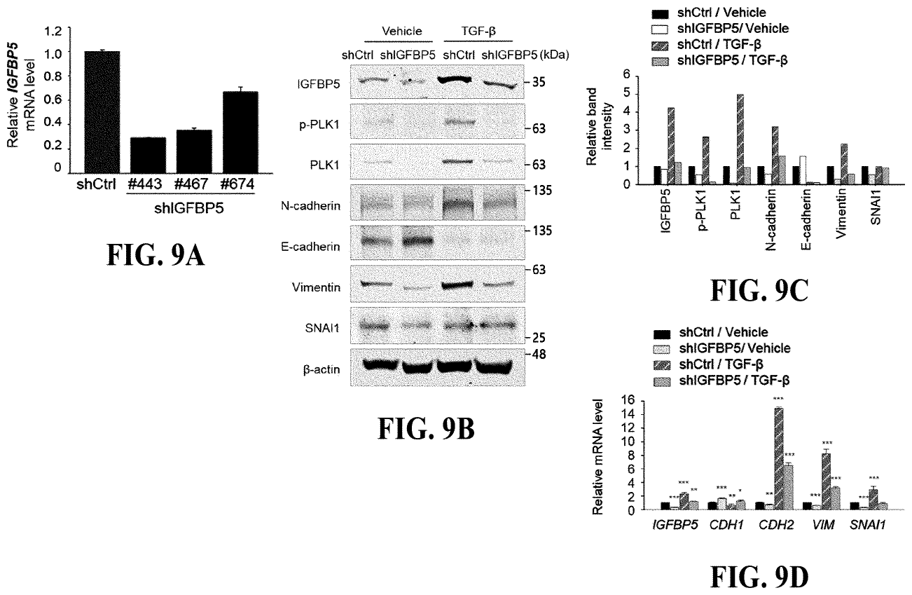

As shown in FIG. 9A, lung cancer cells A549 infected with shIGFBP5 showed decreased mRNA expression of IGFBP5 compared to cells infected with control shRNA (shCtrl), showing that IGFBP5 expression was suppressed. Among the three shRNAs constructed, subsequent experiments were conducted using shRNA targeting #443 site, which exhibited the greatest inhibition effect.

### <Example 10> Confirmation of Inhibition of Epithelial-Mesenchymal Transition by IGFBP5 shRNA

Next, the present inventors suppressed IGFBP5 expression using IGFBP5 shRNA in metastatic cells treated with TGF-β to evaluate epithelial-mesenchymal transition reduction resulting from IGFBP5 expression inhibition and examined epithelial-mesenchymal transition markers at the protein level.

Specifically, lung cancer cells A549 were cultured at 5×10⁴ cells/ml and infected with control virus (shCtrl) or shIGFBP5, then treated with puromycin (Sigma-Aldrich, MO, USA) for 48 hours to select surviving cells, dispensed again at 5×10⁴ cells/ml, and cells were collected after treating with 5 ng/ml TGF-β for 48 hours the next day. The collected cells were treated with 100 μl of lysis solution (0.5% Triton X-100, 20 mM Tris, pH 7.5, 2 mM MgCl₂, 1 mM DTT, 1 mM EGTA, 50 mM beta-glycerophosphate, 25 mM NaF, 1 mM Na₃VO₄, 2 μg/ml leupeptin, 2 μg/ml pepstatin A, 100 μg/ml PMSF, 1 μg/ml antipain) and then protein was quantified. The quantified proteins were electrophoresed through SDS-PAGE, and then an immunoblot was performed using epithelial-mesenchymal transition marker antibodies to confirm the reduction of epithelial-mesenchymal transition processes (FIGS. 9B-9C). Furthermore, mRNA was purified from the collected cells, cDNA was synthesized, and real-time polymerase chain reaction (Real-time PCR) was performed, and it was confirmed that IGFBP5 shRNA reduces epithelial-mesenchymal transition processes in metastatic environments by TGF-β by observing that mesenchymal transition markers, including N-cadherin, Vimentin, SNAI1, etc. decreased and E-Cadherin increased at the mRNA level (FIG. 9D).

### <Example 11> Evaluation of Inhibition Effects on Motility and Invasiveness by IGFBP5 shRNA in Metastatic Environments and Motility and Invasiveness Increase Effects by Phosphorylated Point Mutants

Furthermore, the present inventors demonstrated the effect of IGFBP5 shRNA suppressing cell motility using inserts in metastatic environments.

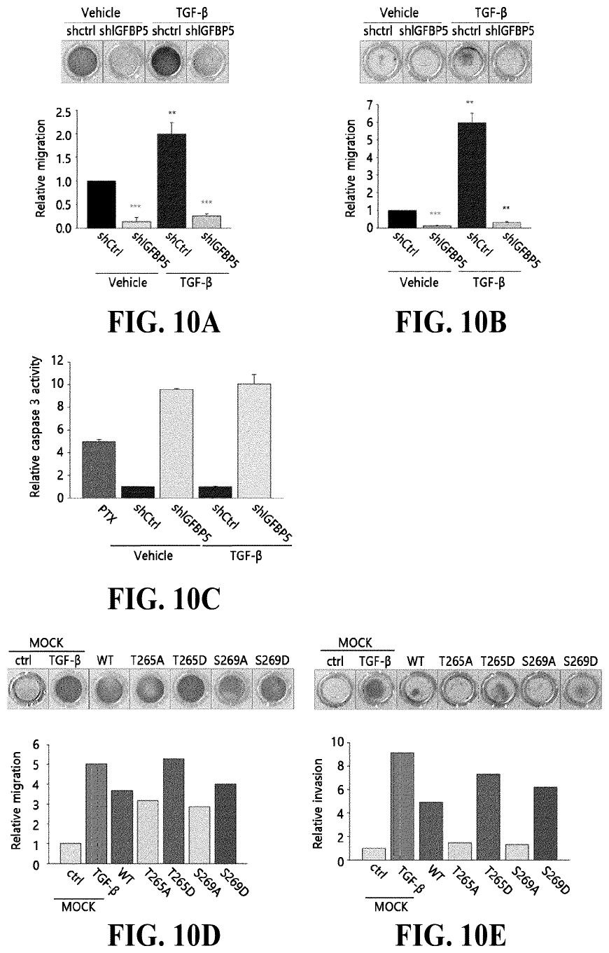

Specifically, lung cancer cells A549 were infected with control shRNA (shCtrl) or IGFBP5 shRNA (shIGFBP5) viruses. The infected cells were diluted in serum-free RPMI (36° C.) at a cell count of 5×10⁴ cells/well and dispensed into 24 well inserts. RPMI (containing 10% FBS) was dispensed at 0.5 ml/well into 24 wells outside the inserts, and after 48 hours, 500 μl of 4% paraformaldehyde was dispensed, followed by washing 3 times with 1×PBS and staining with 0.05% crystal-violet solution for 5 minutes. After 5 minutes, the stained cells were washed 5 times with 1×PBS, and the extent of staining was measured by measuring the wavelength at 590 nm. When the absorbance of the control group was set as 1, the relative absorbance in each experimental group was calculated and displayed as a graph (FIG. 10A).

As a result, as shown in FIG. 10A, compared to cells infected with control virus (shCtrl), when shIGFBP5 was infected to suppress IGFBP5 expression, cell motility greatly decreased. A similar trend was shown when motility was examined after TGF-β treatment (FIG. 10A).

Next, the present inventors demonstrated the effect of IGFBP5 shRNA suppressing cell invasiveness using Matrigel in metastatic environments.

For this experiment, after completely melting Matrigel at 4° C. for 16-20 hours, Matrigel was diluted with cold serum-free MEM (4° C.) to 1 mg/ml. 1 ml of Matrigel mixture (1 mg/ml) was added to 8.0 mm 24 well inserts and solidified in a 37° C. incubator for 12-20 hours. Cells infected with shRNA (shCtrl) or IGFBP5 shRNA (shIGFBP5) viruses were diluted in serum-free RPMI (36° C.) at a cell count of 2×10⁵ cells/well and dispensed into the solidified Matrigel inserts. Warm RPMI (containing 10% FBS) at 36° C. was dispensed at 0.5 ml/well into each well. Thereafter, the medium was changed every 3 days, and the degree of invasion was examined, and on the 7th day when cancer cell invasion was observed to have occurred sufficiently, the medium was removed and washed with 1×PBS, then cells inside the inserts were scraped off with cotton swabs and washed with 1×PBS to remove any remaining cells and Matrigel inside the inserts. 500 μl of 4% paraformaldehyde was dispensed into 24 wells with the outer surface of inserts, incubated at room temperature for 5 minutes, washed 3 times with 1×PBS after 5 minutes, and stained with 0.05% crystal-violet solution for 5 minutes. After 5 minutes, the stained cells were washed 5 times with 1×PBS, and the extent of staining was measured by measuring the wavelength at 590 nm. When the absorbance of the control group was set as 1, the relative absorbance in each experimental group was calculated and displayed as a graph (FIG. 10B).

As a result, as shown in FIG. 10B, compared to cells infected with the control virus (shCtrl), when shIGFBP5 was infected to suppress IGFBP5 expression, cell invasiveness greatly decreased (FIG. 10B).

Lung cancer cells A549 were cultured at 5×10⁴ cells/ml and infected with control virus (shCtrl) or shIGFBP5, then treated with puromycin (Sigma-Aldrich, MO, USA) for 48 hours to select surviving cells, dispensed again at 5×10⁴ cells/ml, and cells were collected after treating with 5 ng/ml TGF-β for 48 hours the next day. The collected cells were treated with 100 μl of lysis solution (0.5% Triton X-100, 20 mM Tris, pH 7.5, 2 mM MgCl₂, 1 mM DTT, 1 mM EGTA, 50 mM beta-glycerophosphate, 25 mM NaF, 1 mM Na₃VO₄, 2 μg/ml leupeptin, 2 μg/ml pepstatin A, 100 μg/ml PMSF, 1 μg/ml antipain) and then protein was quantified. Caspase-3 activity was examined through caspase-3 assay in the quantified proteins, and it was confirmed that caspase-3 activity greatly increased in cells infected with shIGFBP5 (FIG. 10C).

The effect of increasing lung cancer cell motility and invasiveness when IGFBP5 is phosphorylated was demonstrated in addition to when IGFBP5 is suppressed. As a result of evaluating motility and invasiveness of MOCK, IGFBP5 WT, phosphorylated point mutants, and non-phosphorylated point mutants using inserts and Matrigel under the same conditions, it was confirmed that cell motility and invasiveness increased in phosphorylated point mutants compared to WT and non-phosphorylated point mutants (FIGS. 10D-10E).

When the results of FIGS. 10A-10E were combined, it was demonstrated that when IGFBP5 expression is suppressed in lung cancer cells using IGFBP5 shRNA in metastatic environments, epithelial-mesenchymal transition is reduced, and cell motility and invasiveness are also reduced. It was confirmed that cell survival rates decrease as IGFBP5 expression decreases, demonstrating that IGFBP5 affects cell survival. Furthermore, it was demonstrated that when IGFBP5 is phosphorylated, cell motility and invasiveness increase.

### <Example 12> Evaluation of Cell Survival Rate Reduction Effects by IGFBP5 shRNA in Solid Cancers

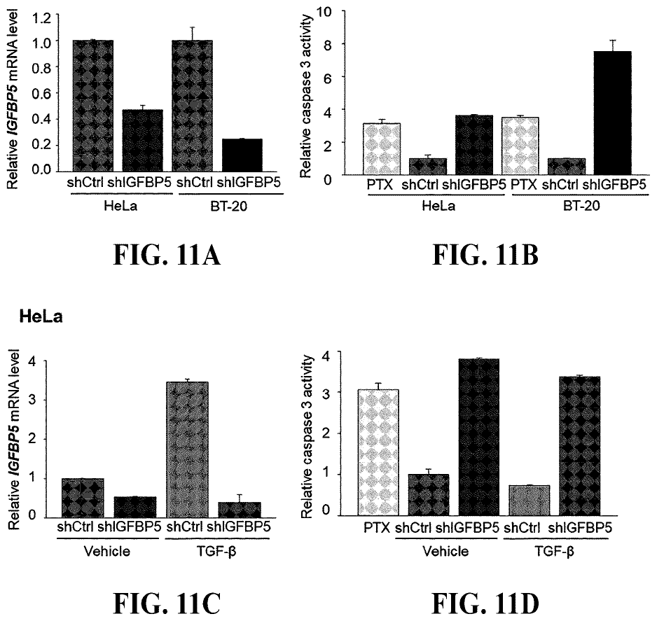

Cervical cancer cells HeLa were cultured at 2×10⁴ cells/ml and breast cancer cells BT-20 were cultured at 4×10⁴ cells/ml, infected with control virus (shCtrl) or shIGFBP5, then treated with puromycin (Sigma-Aldrich, MO, USA) for 48 hours to select surviving cells, dispensed again at 2×10⁴ cells/ml for HeLa cells and 4×10⁴ cells/ml for BT-20 cells, and cells were collected after treating with 5 ng/ml TGF-β for 48 hours the next day. The collected cells were treated with 100 μl of lysis solution (0.5% Triton X-100, 20 mM Tris, pH 7.5, 2 mM MgCl₂, 1 mM DTT, 1 mM EGTA, 50 mM beta-glycerophosphate, 25 mM NaF, 1 mM Na₃VO₄, 2 μg/ml leupeptin, 2 μg/ml pepstatin A, 100 μg/ml PMSF, 1 μg/ml antipain) and then protein was quantified. As shown in FIG. 11A, cervical cancer cells HeLa and breast cancer cells BT-20 infected with shIGFBP5 showed decreased mRNA expression of IGFBP5 compared to cells infected with control shRNA (shCtrl), showing that IGFBP5 expression was suppressed (FIG. 11A). Caspase-3 activity was examined through caspase-3 assay with the quantified proteins, and under conditions where caspase-3 activity increased in cells treated with paclitaxel used as positive control, it was confirmed that caspase-3 activity greatly increased in HeLa cells and BT-20 cells infected with shIGFBP5 (FIG. 11B). Through this, it was demonstrated that IGFBP5 affects the survival of cervical cancer cells.

It was examined whether there were survival rate changes by shIGFBP5 treatment even in metastatic environments induced by TGF-β treatment. As shown in FIG. 11C, cervical cancer cells HeLa infected with shIGFBP5 showed decreased mRNA expression of IGFBP5 compared to cells infected with control shRNA (shCtrl), and IGFBP5 mRNA expression increased in cells infected with shRNA (shCtrl) and then treated with TGF-β (FIG. 11C). Caspase-3 activity was examined through caspase-3 assay with the quantified proteins, and it was confirmed that caspase-3 activity greatly increased in cells infected with shIGFBP5, and even when treated with TGF-β, caspase-3 activity greatly increased in cells infected with shIGFBP5 (FIG. 11D). Through this, it was demonstrated that IGFBP5 affects the survival of cervical cancer cells even in metastatic environments. Through the above content, it was confirmed that survival rates decrease as IGFBP5 expression decreases in lung cancer cells, cervical cancer cells, and breast cancer cells.

While the embodiments have been described with reference to the limited drawings, it will be apparent to one of ordinary skill in the art that various alterations and modifications can be made from the above description. For example, suitable results may be achieved if the described techniques are performed in a different order, and/or if components in a described system, structure, device, or circuit are combined in a different manner, and/or replaced or supplemented by other components or their equivalents.

Therefore, other implementations, other embodiments, and equivalents to the claims are also within the scope of the following claims.

1. An IGFBP5 mutant protein in which an amino acid residue at position 265 and/or 269 of IGFBP5 of SEQ ID NO: 1, threonine, is substituted with glycine, alanine, valine, leucine, isoleucine, proline, phenylalanine, methionine, or tryptophan.

2. The IGFBP5 mutant protein of claim 1,
wherein the IGFBP5 mutant protein comprises an amino acid sequence of SEQ ID NO: 2.

3. The IGFBP5 mutant protein of claim 1,
wherein the IGFBP5 mutant protein comprises an amino acid sequence of SEQ ID NO: 3.

4. The IGFBP5 mutant protein of claim 3,
wherein an amino acid at position 256 of SEQ ID NO: 3 is alanine.

5. The IGFBP5 mutant protein of claim 1,
wherein the IGFBP5 mutant protein comprises an amino acid sequence of SEQ ID NO: 4.

6. The IGFBP5 mutant protein of claim 5,
wherein an amino acid at position 269 of SEQ ID NO: 4 is alanine.

7. A recombinant vector comprising a gene encoding the protein of claim 1.

8. The recombinant vector of claim 7,
wherein the vector is a lentiviral vector.

9. A pharmaceutical composition for cancer prevention or treatment comprising, as an active ingredient, (i) the IGFBP5 mutant protein of claim 1, or (ii) a recombinant vector comprising a polynucleotide encoding the IGFBP5 mutant protein.

10. The pharmaceutical composition for cancer prevention or treatment of claim 9,
wherein the composition inhibits cancer metastasis by reducing motility and invasiveness of cancer cells.

11. The pharmaceutical composition for cancer prevention or treatment of claim 9,
wherein the cancer is a solid cancer.

12. The pharmaceutical composition for cancer prevention or treatment of claim 11,
wherein the solid cancer is one or more cancers selected from the group consisting of glioblastoma, ovarian cancer, bladder cancer, non-functional pituitary adenoma, esophageal cancer, kidney cancer, breast cancer, stomach adenocarcinoma, rectal cancer, cervical cancer, endometrial cancer, ovarian cancer, lung cancer, and pancreatic cancer.

13. The pharmaceutical composition for cancer prevention or treatment of claim 12,
wherein the lung cancer is non-small cell lung cancer.

14. The pharmaceutical composition for cancer prevention or treatment of claim 13,
wherein the non-small cell lung cancer is adenocarcinoma.

15. The pharmaceutical composition for cancer prevention or treatment of claim 9,
wherein the composition further comprises an RNA for inhibiting IGFBP5 expression or a vector expressing the RNA.

16. A pharmaceutical composition for cancer prevention or treatment comprising an RNA for inhibiting IGFBP5 expression or a vector expressing the RNA.

17. The pharmaceutical composition of claim 16,
wherein the RNA for inhibiting IGFBP5 expression is one selected from the group consisting of aptamer, siRNA, shRNA, and miRNA, and
the RNA for inhibiting IGFBP5 expression comprises a base sequence complementary to a base sequence of SEQ ID NO: 11, SEQ ID NO: 15, or SEQ ID NO: 19.

18. The pharmaceutical composition of claim 16,
wherein the RNA for inhibiting IGFBP5 expression is shRNA, and
the shRNA comprises a base sequence complementary to one base sequence selected from the group consisting of SEQ ID NO: 6 to SEQ ID NO: 8.
